(19) United States
(12) Patent Application Publication
(10) Pub. No.: US 2013/0029926 A1
(43) Pub. Date: Jan. 31, 2013

(54) COMPOSITIONS AND METHODS FOR DETERMING CANCER SUSCEPTIBILITY

(71) Applicants: Kirsten Timms, Salt Lake City, UT (US); Jennifer Potter, Salt Lake City, UT (US); Jerry Lanchbury, Salt Lake City, UT (US)

(72) Inventors: Kirsten Timms, Salt Lake City, UT (US); Jennifer Potter, Salt Lake City, UT (US); Jerry Lanchbury, Salt Lake City, UT (US)

(73) Assignee: Myriad Genetics, Inc., Salt Lake City, UT (US)

(21) Appl. No.: 13/508,154

(22) PCT Filed: Nov. 5, 2010
(86) PCT No.: PCT/US10/55708

§ 371 Date: Sep. 19, 2012

Related U.S. Application Data
(60) Provisional application No. 61/258,504, filed on Nov. 5, 2009.

(51) Int. Cl. C12Q 1/68; A61P 35/00; C40B 60/12; A61K 31/704; C40B 30/04; C12M 1/34

(52) U.S. Cl. 514/34; 435/6.11; 435/6.12; 506/9; 435/287.2; 506/39

(57) ABSTRACT

The invention generally relates to a molecular classification of disease and particularly to molecular markers for cancer susceptibility and methods of use thereof. More specifically, the invention relates to the determination, screening, or classification of an individual's genetic risk for breast and ovarian cancer susceptibility.

### CROSS-REFERENCE TO RELATED APPLICATIONS

This application claims priority under 35 U.S.C. §119(e) to U.S. provisional application Ser. No. 61/258,504, filed Nov. 5, 2009, which is hereby incorporated by reference in its entirety.

### FIELD OF THE INVENTION

The invention generally relates to a molecular classification of disease and particularly to molecular markers for cancer susceptibility and methods of use thereof. More specifically, the invention relates to the determination, screening, or classification of an individual's genetic risk for breast and ovarian cancer susceptibility.

### SEQUENCE LISTING

The instant application was filed with a formal Sequence Listing submitted electronically as a text file. This text file, which was named “3316-01-1 WO-2010-11-05-SEQ-LIST-BLC-ST25.txt”, was created on Nov. 5, 2009, and is 333,619 bytes in size. Its contents are incorporated by reference herein in their entirety.

### BACKGROUND OF THE INVENTION

Breast cancer is the most commonly diagnosed cancer in women after nonmelanoma skin cancer, and is the second leading cause of cancer-related deaths. Although less common, ovarian cancer is associated with high morbidity and mortality rates. In fact, among Western women, ovarian cancer ranks as the fourth cause of all cancer-related deaths, and is the most lethal gynecologic malignancy. American Cancer Society, Facts & Figures, 2010. Most breast cancers (70-80%) and ovarian cancers (80-90%) occur in women with no discernable family history of cancer (sporadic cancers). However, significant proportions of breast and ovarian cancers are hereditary.

Hereditary breast and ovarian cancer (“HBOC”) is a syndrome that, in the vast majority of cases, results from mutations in breast and ovarian cancer susceptibility genes, BRCA1 and BRCA2 (referred to collectively as “BRCA”). Consequently, a family history of breast and ovarian cancer is one of the strongest identified risk factors for these cancers, and individuals with a family history of breast and ovarian cancer indicating an increased-risk are usually referred for genetic testing. Genetic testing is now commonly accepted as the most accurate method for diagnosing HBOC.

The identification of BRCA mutations is important to the clinical management of cancer in individuals with an increased predisposition to breast and ovarian cancer and/or families with a history of breast and ovarian cancer. Preventive interventions such as prophylactic surgery (mastectomy and gynecological surgery), chemoprevention and intensive surveillance can reduce the incidence of cancer and mortality. As a result, screening for BRCA mutations is now offered routinely in clinical practice. However, only women with a significant family history of breast and ovarian cancer are generally offered genetic testing.

Screening for inherited breast and ovarian cancer susceptibility is typically a 2-step process: assessment of risk for clinically significant BRCA mutations followed by genetic testing of high-risk individuals. Typically, only high risk individuals undergo genetic testing. Thus, current guidelines recommend testing for mutations only when an individual has personal or family history features suggestive of inherited cancer susceptibility. Unfortunately, current guidelines and screening methods also rely on individuals to self-report a family history of breast and ovarian cancer, which is often inaccurate.

Given the sometimes inconsistent application of family and personal history screening, there is significant need in the art of predictive medicine for improved methods of identifying patients who have an increased likelihood of carrying a BRCA mutation. Furthermore, as deleterious BRCA mutations are associated with predisposition to cancers, particularly breast cancer and ovarian cancer, it is desirable to identify additional naturally existing deleterious BRCA mutations, which may serve as valuable diagnostic markers.

### SUMMARY OF THE INVENTION

It has been discovered that abnormal germline BRCA status is more common than previously thought and that (1) identifying patients as having triple negative breast cancer (“TNBC”), and/or (2) screening the tumors of breast or ovarian cancer patients for abnormal BRCA status, can identify patients who may benefit from germline BRCA testing (e.g., despite lacking a significant personal or family history of cancer). Specifically, we found a higher than expected incidence (19.5%) of germline BRCA mutations in an unselected cohort TNBC patients. We further found a higher than expected incidence (60.7%) of germline BRCA mutations in a cohort of ovarian cancer patients. Thus the invention generally provides methods of identifying BRCA deficient patients, including methods of identifying patients whose germline BRCA status should be determined.

Thus, in one aspect the invention provides methods of identifying patients appropriate for BRCA testing by identifying TNBC patients. In one embodiment the invention provides a method of detecting germline BRCA deficiency comprising identifying a patient with TNBC and determining whether the patient has germline BRCA deficiency. In another embodiment the invention provides a method of identifying a patient whose germline BRCA status should be determined, the method comprising determining whether the patient has TNBC, wherein the presence of TNBC indicates the patient's BRCA status should be determined. In some embodiments the method comprises determining whether a patient has TNBC, wherein the presence of TNBC indicates an increased likelihood of abnormal germline BRCA status. In some embodiments determining whether the patient has TNBC comprises measuring the expression of estrogen receptor, progesterone receptor, and HER2 in a sample from the patient. In some embodiments, if the patient has TNBC, the method further comprises determining the patient's BRCA status (e.g., germline BRCA status).

In another aspect the invention provides methods of identifying patients appropriate for germline BRCA testing by identifying patients with somatic BRCA deficiency. Thus one embodiment of the invention provides a method for determining cancer susceptibility comprising identifying a patient with somatic BRCA deficiency and determining the patient's germline BRCA status, wherein germline BRCA deficiency indicates increased cancer susceptibility. In some embodiments the patient does not have family history of cancer.

BRCA deficiency can be helpful in determining how to treat a patient. For instance, BRCA deficiency can indicate likelihood of response to particular drugs (e.g., DNA damaging agents such as platinum drugs, PARP inhibitors, etc.). Germline BRCA deficiency can also indicate the patient is appropriate for prophylactic medical management (e.g., hormone treatment, prophylactic mastectomy or oophorectomy, etc.). Thus in another aspect the invention provides a method of treating a patient based on whether that patient is BRCA deficient.

In some embodiments the invention provides a method of treating a patient comprising determining whether a patient has TNBC, determining whether the patient is BRCA defective, and selecting a particular treatment course if the patient is BRCA defective. In some embodiments the invention provides a method of treating a patient comprising determining whether a patient has somatic BRCA deficiency, determining whether the patient has germline BRCA deficiency, and selecting a particular treatment course if the patient has germline BRCA deficiency. In some embodiments the particular treatment course comprises DNA-damaging agents, PARP inhibitors, etc. In some embodiments the particular treatment course comprises prophylactic surgery (e.g., mastectomy, oophorectomy, etc.) or prophylactic pharmaceutical treatment (e.g., hormone treatment).

The present invention further provides systems related to the above methods of the invention. In one embodiment the invention provides a system for detecting BRCA deficiency comprising: (1) a sample analyzer for determining whether a tumor sample from a TNBC patient has BRCA deficiency, wherein the sample analyzer contains the sample, mRNA from the sample and expressed from the panel of genes, or cDNA synthesized from said mRNA; (2) a first computer program means for determining BRCA status information (e.g., presence or absence of deleterious mutations, hypermethylation, lowered expression, etc.); and optionally (3) a display means for displaying whether the patient has BRCA deficiency.

BRCA deficiency may be found in various patient tissues, depending on the type of deficiency looked for. In some embodiments, for example, the presence of somatic mutations is determined by analyzing patient tumor tissue. In some embodiments, for example, one determines whether a patient harbors a germ-line mutation by analyzing any non-neoplastic tissue (e.g., blood, blood-derived samples, etc.).

BRCA deficiency of interest according to the present invention can include deleterious mutations (including missense changes, nonsense changes, large rearrangements, etc.), copy number variants (CNVs), lowered (including no) expression (e.g., mRNA expression, protein expression, etc.), methylation amount or pattern that indicates lowered (including no) expression, etc.

Various techniques for determining BRCA deficiency are known to those skilled in the art. In some embodiments the whole genome of one or more cells is determined and the sequence of a BRCA gene found within that genome is analyzed for mutations. In some embodiments a BRCA gene is sequenced in a targeted manner, which may include exon sequencing, sequencing of exons along with at least some amount of flanking intronic sequence, or sequencing of the entire genomic region containing the BRCA gene of interest. Copy number analysis may also be used. In some embodiments large rearrangement analysis is used to determine whether large portions of the BRCA gene (or even the entire gene) have been deleted or duplicated. In some embodiments expression analysis (e.g., measuring mRNA and/or protein expression) is used to determine BRCA deficiency. In some embodiments methylation analysis is used to determine BRCA deficiency.

Novel variants have also been discovered within the BRCA1 gene. Thus one aspect of the invention provides isolated nucleic acids comprising at least one of variant listed in Table 1 or Table 2. Another aspect provides isolated polypeptides comprising at least one of variant listed in Table 1 or Table 2. Yet another aspect provides antibodies that bind selectively to polypeptides comprising at least one of variant listed in Table 1 or Table 2. Still another aspect provides probe sets comprising nucleic acids each comprising at least one of variant listed in Table 1 or Table 2. Another aspect of the invention provides kits comprising the isolated nucleic acids, polypeptides, antibodies, and/or probe sets of the invention.

Another aspect of the invention provides a method of genotyping, comprising determining the genotype at the polymorphism position of a variant listed in Table 1 or Table 2. Genotyping may include determining the nucleotide sequence directly or inferring the nucleotide sequence by determining the amino acid sequence directly. Genotyping may accomplished by various techniques, including but not limited to whole genome sequencing, BRCA gene sequencing, allele-specific oligonucleotide analysis, BRCA protein sequencing, anti-BRCA antibody analysis, etc.

Because the variants listed in Table 1 and Table 2 are deleterious, they may be useful in predicting predisposition to cancer. Thus one aspect of the invention provides a method for determining the cancer susceptibility of a human patient comprising determining the genotype at the polymorphism position of at least one variant listed in Table 1 or Table 2, wherein the presence of at least one of said variants indicates increased cancer susceptibility.

The variants in Table 1 and Table 2 may also be useful in the screening methods of the invention. Thus the invention provides a method of detecting germline BRCA deficiency comprising determining whether a patient has a variant listed in Table 1 or Table 2 in a somatic tissue sample and determining whether the patient has germline BRCA deficiency. The invention further provides a method of detecting germline BRCA deficiency comprising determining whether a patient has a somatic BRCA deficiency and determining whether the patient has germline BRCA deficiency comprising a variant listed in Table 1 or Table 2.

In another aspect of the invention, a method is provided for genotyping BRCA1 to determine whether an individual has a genetic variant or an amino acid variant identified in the present invention. The presence of the variants would indicate a predisposition to cancers including breast cancer and ovarian cancer. In accordance with this aspect of the invention, a sample containing genomic DNA, mRNA, or cDNA of the BRCA1 gene is obtained from the individual to be tested. The genomic DNA, mRNA, or cDNA of the BRCA1 gene in the sample should include at least the nucleotide sequence surrounding the locus of one or more of the above-described genetic variants such that the presence or absence of a particular genetic variant can be determined. Any suitable method known in the art for genotyping can be used for determining the nucleotide(s) at a particular position in the BRCA1 gene. Alternatively, the presence or absence of one or more of the amino acid variants disclosed in Table 1 or Table 2 can also be determined in the BRCA1 protein in a sample isolated from a patient to be tested. The presence of the nucleotide and/or amino acid variants provided in the present invention may be indicative of a likelihood of a predisposition to cancers, e.g., breast cancer and ovarian cancer.

In another aspect of the present invention, a variety of methods are provided for predicting a predisposition to cancer in a patient. The detection step used in such methods can involve the analysis of BRCA1 genomic DNA, cDNA or polypeptides. Analyses of nucleic acids in these instances can involve amplification-based approaches or hybridization-based approaches. Analyses of polypeptides can involve determining whether or not the variant BRCA1 polypeptide is truncated, or contains characteristic epitopes that can be specifically detected with an appropriate antibody.

In another aspect of the invention, a detection kit is also provided for detecting, in an individual, an elevated risk of cancer. In a specific embodiment, the kit is used in determining a predisposition to breast cancer and ovarian cancer. The kit may include, in a partitioned carrier or confined compartment, any nucleic acid probes or primers, or antibodies useful for detecting the BRCA1 variants of the present invention as described above. The kit can also include other reagents such as reverse transcriptase, DNA polymerase, buffers, nucleotides and other items that can be used in detecting the genetic variations and/or amino acid variants according to the method of this invention. In addition, the kit preferably also contains instructions for its use.

The present invention further provides a method for identifying a compound for treating or preventing cancers associated with a BRCA1 genetic variant of the present invention. The method includes screening for a compound capable of selectively interacting with a BRCA1 protein variant of the present invention.

The foregoing and other advantages and features of the invention, and the manner in which the same are accomplished, will become more readily apparent upon consideration of the following detailed description of the invention taken in conjunction with the accompanying examples and drawings, which illustrate preferred and exemplary embodiments.

### BRIEF DESCRIPTION OF THE DRAWINGS

FIG. 1 illustrates the relationship between BRCA1 mutations and BRCA1 gene expression and between BRCA2 mutations and BRCA2 gene expression is shown in panels A and B, respectively.

FIG. 2 illustrates Kaplan-Meier curves showing that BRCA1 and BRCA2 mutations together in ovarian cancer tissue were associated with significantly improved progression-free survival (PFS) time after surgery as compared with BRCA1- and BRCA2-wild type ovarian cancers. Median PFS for BRCA mutants and non-mutants are 20.1 (CI=(15.6, 34.5)) and 13.9 (11.9, 16.9), respectively.

FIG. 3 illustrates Kaplan-Meier curves showing that BRCA1/2 deficiency in ovarian cancer tissue was associated with significantly improved progression-free survival (PFS) time after surgery as compared with other ovarian cancers. Median PFS for cancers with and without BRCA deficiency are 19.7 (CI=(15.7, 31.6)) and 13.8 (11.7, 17.7), respectively.

FIG. 4 illustrates the relationship between BRCA deficiency and RFS and OS in TNBC patients.

FIG. 5 illustrates general features of a computer system of the invention.

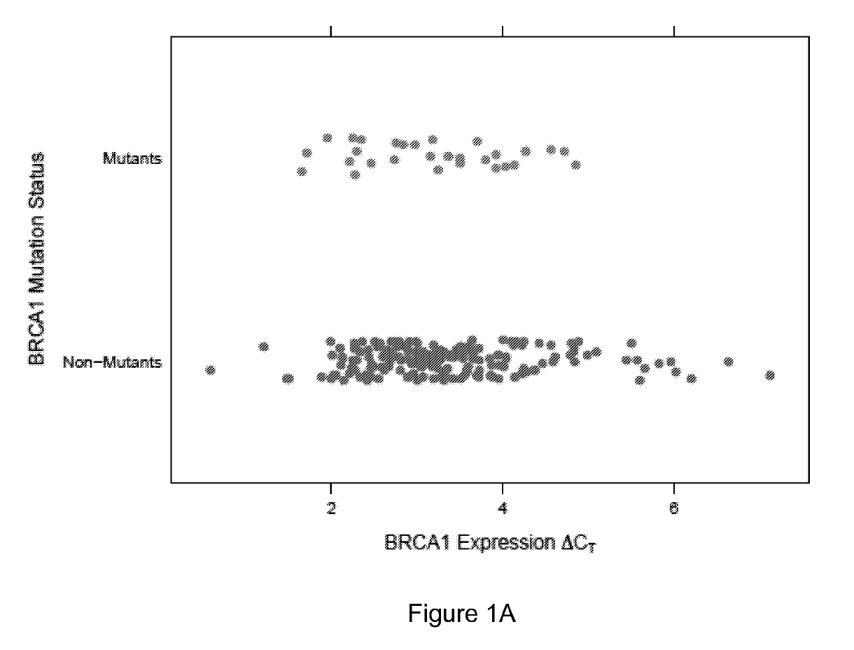

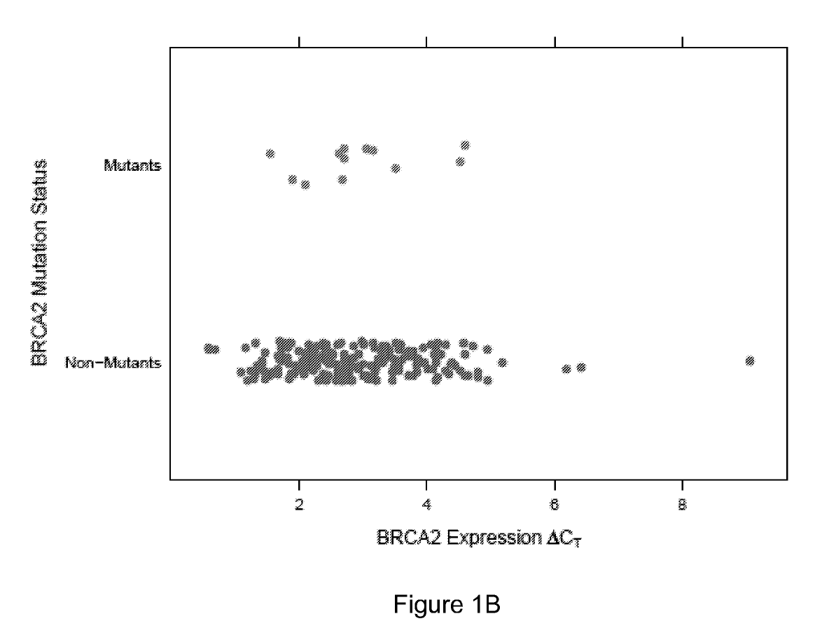

### DETAILED DESCRIPTION OF THE INVENTION

It has been discovered that abnormal germline BRCA status is more common than previously thought and that (1) identifying patients as having triple negative breast cancer (“TNBC”), and/or (2) screening the tumors of breast or ovarian cancer patients for abnormal BRCA status, can identify patients appropriate for germline BRCA testing (e.g., despite lacking a significant personal or family history of cancer). Thus the invention generally provides methods of identifying BRCA deficient patients, including methods of identifying patients whose germline BRCA status should be determined.

As shown in Example 1, we have discovered that nearly 20% of unselected TNBC patients have germline BRCA deficiency. This compares with a germline BRCA deficiency rate of approximately 5-10% in the general breast cancer population. Medical society guidelines suggest BRCA genetic testing for individuals with a risk of BRCA deficiency of 15-20% or greater. These risks of BRCA deficiency are typically assessed by analyzing a patient's personal and family history of cancer. We have surprisingly found that TNBC patients should be considered for BRCA genetic testing—i.e., TNBC is itself a sufficiently significant risk factor for BRCA deficiency to warrant genetic BRCA testing—regardless of personal or family cancer history (including in the absence of significant personal or family cancer history).

Thus, in one aspect the invention provides methods of identifying patients appropriate for BRCA testing by identifying TNBC patients. In one embodiment the invention provides a method of detecting BRCA deficiency comprising identifying a patient with TNBC and determining whether the patient has BRCA deficiency (e.g., somatic or germline). In another embodiment the invention provides a method of identifying a patient whose BRCA status should be determined comprising determining whether the patient has TNBC, wherein the presence of TNBC indicates the patient's BRCA status (e.g., somatic or germline) should be determined. In some embodiments the BRCA status to be determined is the somatic BRCA status of the tumor. In other embodiments the BRCA status to be determined is the germline BRCA status of the patient.

As shown in Example 2, somatic BRCA deficiency is a predictor of germline deficiency. Specifically, over 60% of samples having somatic BRCA deficiency were also found to harbor germline deficiency. Thus, in some embodiments BRCA deficiency is first assessed in somatic (i.e., tumor) tissue and then, if there is somatic BRCA deficiency, germline assessment is done. In some embodiments the invention provides a method comprising determining whether a patient's tumor sample has BRCA deficiency and, if there is somatic BRCA deficiency, determining whether the patient has germline BRCA deficiency. In some embodiments the invention provides a method comprising identifying a patient having TNBC, determining whether a patient's tumor sample has BRCA deficiency and, if there is somatic BRCA deficiency, determining whether the patient has germline BRCA deficiency. In some embodiments the invention provides a method comprising determining whether a patient's tumor sample has BRCA deficiency, and, if there is somatic BRCA deficiency, (1) determining whether the patient has TNBC and (2) determining whether the patient has germline BRCA deficiency.

In some embodiments the patient is identified as lacking one or more (or any) significant risk factors for germline BRCA deficiency. In other embodiments the patient is identified as lacking any significant personal and/or family history of cancer. “Significant family history of cancer” and “significant personal history of cancer” are well-known terms in the art. Significant risk factors for germline BRCA deficiency are also well-known and well-documented in the art and are generally features of a patient's personal or family history (including ethnic background) that suggest an increased probability of carrying a germline BRCA deficiency. Various guidelines have been devised and are used by healthcare professionals to determine whether an individual has a significant risk factor for germline BRCA deficiency. These include guidelines of American Gastroenterological Association; American Society of Breast Surgeons; American Society of Clinical Oncology; American Society of Colon & Rectal Surgeons; Oncology Nursing Society; Society of Gynecologic Oncologists (e.g., women with breast cancer at ≦40 years, women with bilateral breast cancer (particularly if the first cancer was at ≦50 years); women with breast cancer at ≦50 years and a close relative with breast cancer at ≦50 years; women of Ashkenazi Jewish ancestry with breast cancer at ≦50 years; women with breast or ovarian cancer at any age and two or more close relatives with breast cancer at any age (particularly if at least one breast cancer was at ≦50 years); unaffected women with a first or second degree relative that meets one of the above criteria), etc. Other widely accepted criteria include individuals with a personal or family history of breast cancer before age 50 or ovarian cancer at any age; individuals with two or more primary diagnoses of breast and/or ovarian cancer; individuals of Ashkenazi Jewish descent with a personal or family history of breast cancer before age 50 or ovarian cancer at any age; male breast cancer patients. A patient lack a significant history of cancer when one or more (or all) of these criteria are not met.

The American College of Obstetricians and Gynecologists (ACOG), for example, identifies women who have more than a 20%-25% chance of having an inherited predisposition to breast or ovarian cancer as being appropriate for BRCA testing (i.e., having a significant personal or family history of cancer). These women include those with any of the following significant risk factors for germline BRCA deficiency:
Women with a personal history of both breast cancer and ovarian cancer.
Women with ovarian cancer and a close relative—defined as mother, sister, daughter, grandmother, granddaughter, aunt—with ovarian cancer, premenopausal breast cancer, or both.
Women of Ashkenazi Jewish decent with breast cancer who were diagnosed at age 40 or younger or who have ovarian cancer.
Women with breast cancer at 50 or younger and who have a close relative with ovarian cancer or male breast cancer at any age.
Women with a close relative with a known BRCA mutation.

ACOG further identifies women with a 5%-10% chance of having hereditary risk as also potentially being appropriate for BRCA testing (i.e., having a significant personal or family history of cancer). These women include those with any of the following significant risk factors for germline BRCA deficiency:
Women with breast cancer by age 40.
Women with ovarian cancer, primary peritoneal cancer, or fallopian tube cancer or high grade, serous histology at any age.
Women with cancer in both breasts (particularly if the first cancer was diagnosed by age 50).
Women with breast cancer by age 50 and a close relative with breast cancer by age 50.
Women with breast cancer at any age and two or more close relatives with breast cancer at any age (particularly if at least one case of breast cancer was diagnosed by age 50).
Unaffected women with a close relative that meets one of the previous criteria.

The American Society of Breast Surgeons recommends BRCA testing for women with any of the following significant risk factors for germline BRCA deficiency:
early onset breast cancer (diagnosed before age 50);
two primary breast cancers, either bilateral or ipsilateral;
a family history of early onset breast cancer;
male breast cancer;
a personal or family history of ovarian cancer (particularly non-mucinous types);
Ashkenazi (Eastern European) Jewish heritage; or
a previously identified BRCA1 or BRCA2 mutation in the family.

In Example [X], the inventors tested a group of unselected TNBC patients and found high incidence of germline BRCA deficiency. In other words, the TNBC patients had not been selected (i.e., identified) as having any significant risk factors for germline BRCA deficiency. Thus, in some embodiments the patient has not been identified as having any of the above significant risk factors for germline BRCA deficiency (e.g., not identified as having any of the above risk factors). In other embodiments of the invention one may positively identify a patient as lacking any significant risk factors for germline BRCA deficiency (e.g., none of the above risk factors).

In some embodiments the patient lacks particular significant risk factors for germline BRCA deficiency. Thus in some embodiments the patient is not of Ashkenazi Jewish descent. In some embodiments the patient's cancer was diagnosed after 40, 45, 50, 55, 60, 65 or more years of age. In some embodiments the patient has not been diagnosed with ovarian cancer, primary peritoneal cancer, or fallopian tube cancer or high grade, serous histology. In some embodiments the patient has no close relatives diagnosed with ovarian cancer, primary peritoneal cancer, or fallopian tube cancer or high grade, serous histology. In some embodiments the patient has no close male relative with breast cancer. In some embodiments the patient has not been diagnosed with two primary breast cancers, either bilateral or ipsilateral. In some embodiments the patient has no close relatives with a known germline BRCA deficiency.

There are various risk assessment models and medical society guidelines (e.g., the guidelines listed above) that can identify patients having or not having a significant personal and/or family history of cancer. Well-known statistical risk models include the Gail, Ford, Claus and Tyrer-Cuzick models (Amir et al., J. MED. GENET. (2003) 40:807-814) as well as BRCAPRO and MYRIADII (Weinstock et al., PROC. AM. SOC. CLIN. ONCOL. 20:2001 (abstr 1676)). These models generally give a percentage probability of a particular patient developing breast cancer and clinicians will often use these percentages in determining which patients have a high enough risk to warrant BRCA testing. Thus, in some embodiments of the invention the patient is identified as not having a significant personal or family history of cancer if the patient's risk of cancer according one or more of these models is 30%, 25%, 20%, 15%, 10%, 9%, 8%, 7%, 6%, 5%, 4%, 3%, 2%, 1% or less.

“Triple negative breast cancer” and “TNBC” are well-known terms in the art. TNBC generally does not express estrogen receptor (ER) or progesterone receptor (PR) and does not overexpress HER2/neu. Methods for determining triple negative status are well-known in the art and may include immunohistochemistry of preserved tumor samples using antibodies against ER, PR and/or HER2 protein, mRNA analysis for ER, PR and/or HER2 transcripts, genetic analysis to detect amplification of the HER2 gene, etc.

In some embodiments the method comprises determining whether a patient has TNBC. In some embodiments determining whether the patient has TNBC comprises measuring the expression of estrogen and/or progesterone receptor, and/or (1) measuring HER2 expression and/or (2) measuring HER2 gene amplification. Thus in some embodiments the invention provides a method comprising (1) determining whether a patient has TNBC by measuring the expression of estrogen and/or progesterone receptor, and/or (a) measuring HER2 expression and/or (b) measuring HER2 gene amplification; (2) determining whether a tumor sample from the patient has BRCA deficiency; and, if the tumor sample has BRCA deficiency, optionally (3) determining whether the patient has germline BRCA deficiency.

In another aspect the invention provides a method for determining cancer susceptibility comprising identifying a patient with BRCA deficiency in somatic tissue and determining the patient's germline BRCA status, wherein a germline BRCA deficiency indicates increased cancer susceptibility. As used herein, the “status” of a gene means the presence, absence, or extent/level of some physical, chemical, or genetic characteristic of the gene or its expression product(s). Such characteristics include, but are not limited to, mutations, copy number variants (CNVs), methylation, expression levels, activity levels, etc. These may be assayed directly (e.g., by assaying a gene's expression level) or determined indirectly (e.g., assaying the level of a gene or genes whose expression level is correlated to the expression level of the gene of interest). Those skilled in the art are familiar with various techniques for determining the status of a gene or protein including, but not limited to, whole genome or gene-specific sequencing, locus-specific genotyping (e.g., SNP arrays), large-rearrangement analysis, CNV analysis, microarray mRNA expression analysis, quantitative real-time PCR (qRT-PCR, e.g., TaqMan), immunoanalysis (e.g., ELISA, immunohistochemistry), etc. The methods of the invention may be practiced independent of the particular technique used. In some embodiments, multiple techniques are used to confirm a gene's status (see, e.g., Example 1).

“BRCA deficiency” in a sample means the sample contains (1) a BRCA gene containing a deleterious mutation (including large rearrangements such as large deletions, duplications, etc.), (2) a BRCA gene with higher than normal levels of methylation that results in lowered expression of the gene, (3) lower than normal levels of mRNA expression of a BRCA gene, or (4) lower than normal levels of protein expression of a BRCA protein. For example, “elevated” means that one or more of the above characteristics (e.g., methylation) is higher than normal levels. Generally this means an increase in the characteristic (e.g., expression) as compared to an index value. Conversely, “low” means that one or more of the above characteristics (e.g., expression) is lower than normal levels. Generally this means a decrease in the characteristic (e.g., expression) as compared to an index value. In this context, a “negative status” generally means the characteristic is absent or undetectable. For example, BRCA status is negative if BRCA nucleic acid and/or protein is absent or undetectable in a sample. However, negative BRCA status also includes a mutation or copy number variation in BRCA.

Often testing somatic tissue will reveal a deficiency for a BRCA gene and this will reflex into determining whether there is germline BRCA deficiency. Thus another aspect of the invention provides a method for determining the cancer susceptibility of a patient comprising detecting BRCA deficiency in a somatic tissue sample from the patient (e.g., in the patient's tumor tissue) and determining whether the patient has germline BRCA deficiency, wherein germline BRCA deficiency indicates increased cancer susceptibility. As used herein, “somatic” has its conventional meaning in the art and is opposed to “germline,” which also has its conventional meaning in the art. Generally speaking, a somatic mutation is one that appears in a specific cell or tissue of an organism but not throughout every cell of the organism, which would in turn be germline. Thus determining the status of a gene in somatic tissue refers to determining status in a tissue that may or may not differ in its genetic makeup from germline cells (e.g., tumor tissue). Conversely, determining germline status refers to determining status in a cell or tissue that is expected to have the same genetic makeup as the rest of the organism (e.g., blood cells) and thus be representative of the inherited genetic makeup of the organism.

Thus in some embodiments the patient to be assessed by the methods of the invention does not have a significant family history of cancer. In some embodiments the patient has neither a personal nor a significant family history of cancer. In such cases an individual who would otherwise not be indicated for germline BRCA testing may actually benefit if the individual's tumor has an abnormal BRCA status. Thus the invention provides a method of identifying a patient who might benefit from germline BRCA testing comprising determining whether a tumor in the patient has abnormal BRCA status, wherein abnormal BRCA status indicates the patient might benefit from germline BRCA testing.

One aspect of the invention provides methods of treatment (e.g., computer-implemented methods) involving determining BRCA status in a tumor and then assessing whether the patient should undergo germline BRCA testing based on whether the tumor has an abnormal BRCA status. Thus in one embodiment the invention provides a method (including a computer-implemented method) comprising accessing information on the BRCA status of a patient's tumor sample, querying whether the tumor sample has abnormal BRCA status, outputting or displaying the result of the query, and optionally recommending germline BRCA testing if the tumor sample has an abnormal BRCA status.

Abnormal germline BRCA status can indicate more than just cancer susceptibility. For example, abnormal germline BRCA status can indicate likelihood of response to particular drugs once cancer develops. Examples include DNA-damaging agents (e.g., platinum drugs such as cisplatin, oxaliplatin, carboplatin, etc.) and poly (ADP-ribose) polymerase (PARP) inhibitors. Thus the invention provides a method of predicting response to cancer therapy comprising determining the status of a BRCA gene in a somatic tissue sample from a patient, determining the germline BRCA status of the patient if said somatic tissue sample shows abnormal BRCA status, and prescribing, administering, or recommending a DNA-damaging therapeutic agent or a PARP inhibitor for any subsequent cancer if the germline BRCA status is abnormal. This aspect of the invention is particularly useful in predicting therapy efficacy in any cancers that might appear after the initial cancer is treated since these subsequent cancers are likely to arise at least in part from the abnormal germline BRCA status and thus respond well to these particular drugs.

Abnormal status may be found in various patient tissues, depending on the status indicator to be analyzed. In some embodiments, for example, the presence of somatic mutations is determined by analyzing patient tumor tissue. In other embodiments, for example, one determines germline BRCA status analyzing any non-neoplastic tissue (e.g., blood, blood-derived samples, etc.).

An abnormal status of interest according to the present invention can include deleterious mutations (including missense changes, nonsense changes, large rearrangements, etc.), CNVs, lowered (including no) expression (e.g., mRNA expression, protein expression, etc.), amount or pattern of methylation that indicates lowered (including no) expression, etc.

Various techniques for determining BRCA status are known to those skilled in the art. In some embodiments the whole genome of one or more cells is determined and the sequence of a BRCA gene found within that genome is analyzed for mutations, deletions, amplifications, etc. In some embodiments a BRCA gene is specifically sequenced, which may include exon sequencing, sequencing of exons along with at least some amount of flanking intronic sequence, or sequencing of the entire genomic region containing the BRCA gene of interest. Copy number analysis may also be used. In some embodiments large rearrangement analysis is used to determine whether large portions of the BRCA gene (or even the entire gene) have been deleted or duplicated. This will often involve microarray analysis (e.g., SNP array) in order to determine copy number of the presence or large deletions. In some embodiments methylation analysis is used to determine BRCA status. In some embodiments, specific mutations are searched for (e.g., founder Ashkenazi mutations known in the art). This will often involve TaqMan™ analysis to find the mutation or some allele-specific oligonucleotide hybridization technique that can discriminate mutant from wild-type. This list of techniques for determining BRCA status is not exhaustive; those skilled in the art are familiar with various routine techniques (such as those discussed in more detail below) that will serve this purpose.

In some embodiments the invention provides a method of treating a patient comprising determining whether a patient has TNBC, determining whether the patient is BRCA defective, and selecting a particular treatment course if the patient is BRCA defective. In some embodiments the invention provides a method of treating a patient comprising determining whether a patient has somatic BRCA deficiency, determining whether the patient has germline BRCA deficiency, and selecting a particular treatment course if the patient has germline BRCA deficiency. In some embodiments the particular treatment course comprises DNA-damaging agents, PARP inhibitors, etc. In some embodiments the particular treatment course comprises prophylactic surgery (e.g., mastectomy, oophorectomy, etc.) or prophylactic pharmaceutical treatment (e.g., hormone treatment).

### 2. Systems

One aspect of the present invention provides systems related to the above methods of the invention. Embodiments of this aspect generally provide a system for determining whether a patient has BRCA deficiency, increased susceptibility to breast or ovarian cancer, improved prognosis with a particular treatment, etc. Generally speaking, the system comprises (1) a sample analyzer for determining, e.g., whether a patient sample is a TNBC sample, whether a patient sample is BRCA deficient, etc.; (2) computer program means for receiving, storing, and/or retrieving a patient's information regarding TNBC status, BRCA status, etc.; (3) computer program means for querying this patient information; (3) computer program means for concluding, based on this patient data, e.g., whether the patient or a tumor sample from the patient is BRCA deficient, whether there is an increased susceptibility to breast or ovarian cancer, etc.; and optionally (4) computer program means for outputting/displaying this conclusion. In some embodiments this means for outputting the conclusion may comprise a computer program means for informing a health care professional of the conclusion.

In one embodiment the invention provides a system for detecting BRCA deficiency comprising: (1) a sample analyzer for determining whether a tumor sample from a TNBC patient has BRCA deficiency, wherein the sample analyzer contains the sample, mRNA from the sample and expressed from the panel of genes, or cDNA synthesized from said mRNA; and (2) a computer program means for determining BRCA status information (e.g., presence or absence of deleterious mutations, hypermethylation, lowered expression, etc.). In some embodiments, the system further comprises a display module displaying the BRCA status (germline or somatic), optionally along with TNBC status, of the sample.

The sample analyzer can be any instrument useful in determining gene expression, including, e.g., a sequencing machine, a real-time PCR machine, a microarray instrument, etc. In some embodiments the sample analyzer sequences the BRCA genes in the sample. In some embodiments the sample analyzer sequences the entire genome, exome, or transcriptome of the sample and the computer program means for determining BRCA status information analyzes the data produced by the sample analyzer corresponding to the BRCA genes.

In some embodiments the system comprises a plurality of sample analyzers, each capable of performing a separate molecular analysis. In some embodiments the system comprises a sample analyzer capable of determining TNBC status of a sample (e.g., IHC analyzer, ELISA analyzer, etc.) as well as a sample analyzer capable of sequencing the BRCA genes in a sample. Such sequencing analyzers are often also capable of measuring mRNA expression, or mRNA expression analysis can be done by yet another sample analyzer.

As is known to those skilled in the art, the various components of the system need not be physically attached. In some embodiments the sample analyzer transmits the results of its analysis (e.g., raw data) to the computer means, and/or the computer means optionally transmits the results of its analysis to the display module, via an Internet connection, radio or satellite transmission, etc.

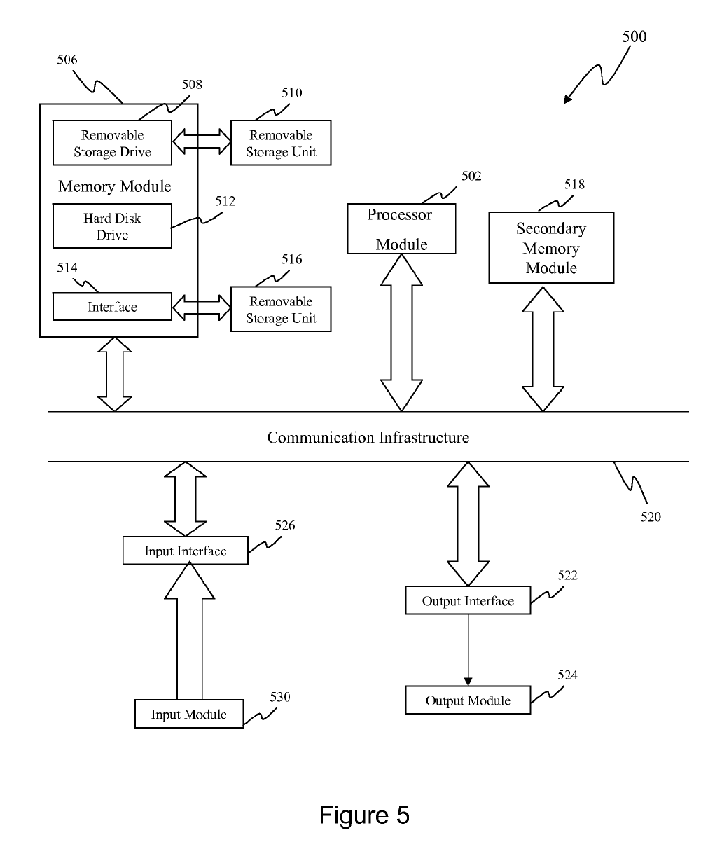

One example of a computer system is the computer system [500] illustrated in FIG. 5. Computer system [500] may include at least one input module [530] for entering patient data into the computer system [500]. The computer system [500] may include at least one output module [524] for indicating whether a patient has an increased or decreased likelihood of response and/or indicating suggested treatments determined by the computer system [500]. Computer system [500] may include at least one memory module [506] in communication with the at least one input module [530] and the at least one output module [524].

The at least one memory module [506] may include, e.g., a removable storage drive [508], which can be in various forms, including but not limited to, a magnetic tape drive, a floppy disk drive, a VCD drive, a DVD drive, an optical disk drive, etc. The removable storage drive [508] may be compatible with a removable storage unit [510] such that it can read from and/or write to the removable storage unit [510]. Removable storage unit [510] may include a computer usable storage medium having stored therein computer-readable program codes or instructions and/or computer readable data. For example, removable storage unit [510] may store patient data. Example of removable storage unit [510] are well known in the art, including, but not limited to, floppy disks, magnetic tapes, optical disks, and the like. The at least one memory module [506] may also include a hard disk drive [512], which can be used to store computer readable program codes or instructions, and/or computer readable data.

In addition, as shown in FIG. 5, the at least one memory module [506] may further include an interface [514] and a removable storage unit [516] that is compatible with interface [514] such that software, computer readable codes or instructions can be transferred from the removable storage unit [516] into computer system [500]. Examples of interface [514] and removable storage unit [516] pairs include, e.g., removable memory chips (e.g., EPROMs or PROMs) and sockets associated therewith, program cartridges and cartridge interface, and the like. Computer system [500] may also include a secondary memory module [518], such as random access memory (RAM).

Computer system [500] may include at least one processor module [502]. It should be understood that the at least one processor module [502] may consist of any number of devices. The at least one processor module [502] may include a data processing device, such as a microprocessor or microcontroller or a central processing unit. The at least one processor module [502] may include another logic device such as a DMA (Direct Memory Access) processor, an integrated communication processor device, a custom VLSI (Very Large Scale Integration) device or an ASIC (Application Specific Integrated Circuit) device. In addition, the at least one processor module [502] may include any other type of analog or digital circuitry that is designed to perform the processing functions described herein.

As shown in FIG. 5, in computer system [500], the at least one memory module [506], the at least one processor module [502], and secondary memory module [518] are all operably linked together through communication infrastructure [520], which may be a communications bus, system board, cross-bar, etc.). Through the communication infrastructure [520], computer program codes or instructions or computer readable data can be transferred and exchanged. Input interface [526] may operably connect the at least one input module [526] to the communication infrastructure [520]. Likewise, output interface [522] may operably connect the at least one output module [524] to the communication infrastructure [520].

The at least one input module [530] may include, for example, a keyboard, mouse, touch screen, scanner, and other input devices known in the art. The at least one output module [524] may include, for example, a display screen, such as a computer monitor, TV monitor, or the touch screen of the at least one input module [530]; a printer; and audio speakers. Computer system [500] may also include, modems, communication ports, network cards such as Ethernet cards, and newly developed devices for accessing intranets or the internet.

The at least one memory module [506] may be configured for storing patient data entered via the at least one input module [530] and processed via the at least one processor module [502]. Patient data relevant to the present invention may include sequence data, expression level data, copy number data, etc. for ER, PR, HER2 and/or one or both of the BRCA genes. Any other patient data a physician might find useful in making treatment decisions/recommendations may also be entered into the system, including but not limited to age, gender, and race/ethnicity and lifestyle data such as diet information. Other possible types of patient data include symptoms currently or previously experienced, patient's history of illnesses, medications, and medical procedures.

The at least one memory module [506] may include a computer-implemented method stored therein. The at least one processor module [502] may be used to execute software or computer-readable instruction codes of the computer-implemented method. The computer-implemented method may be configured to, based upon the patient data, indicate whether the patient has an increased likelihood of recurrence, progression or response to any particular treatment, generate a list of possible treatments, etc.

In certain embodiments, the computer-implemented method may be configured to identify a patient as having an increased likelihood of having a BRCA deficiency or, if the patient has such a deficiency, an increased susceptibility of breast or ovarian cancer. For example, the computer-implemented method may be configured to inform a physician that a particular patient has a BRCA deficiency or, if the patient has such a deficiency, an increased susceptibility of breast or ovarian cancer. Alternatively or additionally, the computer-implemented method may be configured to actually suggest a particular course of treatment (e.g., prophylactic treatment such as surgery, treatment with DNA-damaging agents or PARP inhibitors, etc.) based on the answers to/results for various queries.

The computer-based analysis function can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™—or JavaScript™-enabled browsers such as HotJava™, Microsoft™ Explorer™, or Netscape™ can be used. When active content web pages are used, they may include Java™ applets or ActiveX™ controls or other active content technologies.

The analysis function can also be embodied in computer program products and used in the systems described above or other computer- or internet-based systems. Accordingly, another aspect of the present invention relates to a computer program product comprising a computer-usable medium having computer-readable program codes or instructions embodied thereon for enabling a processor to carry out gene status analysis. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

### 3. Nucleotide and Amino Acid Variants

In accordance with the present invention, analysis of the nucleotide sequence of tumor and genomic DNA corresponding to the BRCA genes of specific human patients has led to the discovery of a number of mutant BRCA1 alleles relative to the reference sequence provided by GenBank Accession No. U14680. The term “reference sequence” refers to a polynucleotide or polypeptide sequence known in the art, including those disclosed in publicly accessible databases, e.g., GenBank, or a newly identified gene sequence, used simply as a reference with respect to the nucleotide variants provided in the present invention. The nucleotide or amino acid sequence in a reference sequence is contrasted to the alleles disclosed in the present invention having newly discovered nucleotide or amino acid variants. Another reference sequence was described by Smith and coworkers with the complete genomic sequence of a 117 kilobase region of human DNA containing the BRCA1 gene, and deposited the nucleotide sequence of the genomic DNA in the GenBank under the Accession Number L78833.1 (Smith et al., Genome Res., 6:1029-1049 (1996)).

The genetic variants are summarized in Table 1 and Table 2 below. The BRCA1/BRCA2 numbering for the traditional mutation nomenclature used in BIC Database is based on reference sequences as stated above (i.e., GenBank Accession No. U14680) where the A of the ATG translation initiation codon is at the position of 120 of BRCA1. The approved systematic nomenclature follows the rule where the A of the ATG translation initiation codon is +1. The approved systemic nomenclature is used in parenthesis.

TABLE 1

| | | | Mutation | | |
|---|---|---|---|---|---|
| | | | Position | | |
| | SEQ | | from | | |
| Mutation | ID | Mutation | Start | AA | |
| No. | NOs | (all BRCA1) | Codon | Change | Significance |
| 1 | 3-6, 24, | (978delAACAG) | 859 | Frame-Shift | deleterious |
| | 33 | | | | |
| 2 | 7-10, 26, | (2565delCATGGTTGTTC) | 2446 | Frame-Shift | deleterious |
| | 34 | | | | |
| 3 | 11-14, | (2625G > T) | 2506 | E836* | deleterious |
| | 28, 35 | | | | |
| 4 | 15-18, | (4944G > T) | 4825 | E1609* | deleterious |
| | 30, 36 | | | | |
| 5 | 19-22, | (5293delAAAG) | 5294 | Frame-Shift | deleterious |
| | 32, 37 | | | | |

TABLE 2

| Germline | Somatic |
|---|---|
| BRCA1 187delAG (n = 3) | BRCA1 S451X (1471C > G) |
| BRCA1 2795delAAAG | |
| BRCA1 M1775R (5443T > G) | |
| BRCA1 3829delT | |
| BRCA1 C61G (300T > G) | |
| BRCA1 E29X (204G > T) | |
| BRCA1 S451X (1471C > G) | |
| BRCA1 E1134X (3519G > T) | |
| BRCA1 Del Exon 17 | |
| BRCA2 5804del4 | |
| BRCA2 5578delAA | |
| BRCA2 E3111X (9559G > T) | |

The deleterious classification includes all nonsense mutations and all frame-shift mutations that begin at or before the last known nonsense or frame-shift mutation shown to cosegregate with disease. In addition, specific missense mutations and noncoding intervening sequence (IVS) mutations are recognized as deleterious on the basis of data derived from linkage analysis of high-risk families, functional assays, biochemical evidence, and/or demonstration of abnormal mRNA transcript processing. Suspected deleterious are genetic variants for which all of the available evidence indicates a very strong likelihood that the mutation is harmful or deleterious but whose effect on protein function cannot easily be determined. A suspected deleterious result typically is treated clinically as a deleterious (mutation positive) result.

The genetic variants are indicated in Table 1 and Table 2 by their positions and nucleotide and/or amino acid changes. The nucleotide sequences surrounding each of the genetic variants are provided in SEQ ID NOs:3-22 as indicated in Table 1 above. However, it is noted that the nucleotide variants of the present invention are by no means limited to be only in the context of the sequences in the sequence listings or the particular position referred to herein. Rather, it is recognized that GenBank sequences may contain unrecognized sequence errors only to be corrected at a later date, and additional gene variants may be discovered in the future. The present invention encompasses nucleotide variants as referred to in Table 1 or Table 2 irrespective of such sequence contexts. Indeed, even if the GenBank entries referred to herein are changed based on either error corrections or additional variants discovered, skilled artisans apprised of the present disclosure would still be able to determine or analyze the nucleotide variants of the present invention in the new sequence contexts.

The terms “genetic variant,” “nucleotide variant” and “mutations” are used herein interchangeably to refer to changes or alterations to the reference human genomic DNA or cDNA sequences at a particular locus, including, but not limited to, nucleotide base deletions, insertions, inversions, and substitutions in the coding and non-coding regions. Deletions may be of a single nucleotide base, a portion or a region of the nucleotide sequence of the gene, or of the entire gene sequence. Insertions may be of one or more nucleotide bases. The “genetic variant” or “nucleotide variants” may occur in transcriptional regulatory regions, untranslated regions of mRNA, exons, introns, or exon/intron junctions. The “genetic variant” or “nucleotide variants” may or may not result in stop codons, frame shifts, deletions of amino acids, altered gene transcript splice forms or altered amino acid sequence.

As used herein, the term “amino acid variant” is used to refer to an amino acid change to a reference human protein sequence resulting from “genetic variants” or “nucleotide variants” to the reference human gene encoding the reference protein. The term “amino acid variant” is intended to encompass not only single amino acid substitutions, but also amino acid deletions, insertions, and other significant changes of amino acid sequence in the reference protein.

Accordingly, the present invention provides an isolated nucleic acid comprising at least one of the nucleotide variants as summarized in Table 1 and Table 2. The term “nucleic acid” is inclusive and may be in the form of either double-stranded or single-stranded nucleic acids, and a single strand can be either of the two complementing strands. The isolated nucleic acid can be naturally existing genomic DNA, mRNA or cDNA. In one embodiment, the isolated nucleic acid comprises a nucleotide sequence according to SEQ ID NO:3-32 containing one or more exonic nucleotide variants of Table 1 and Table 2, or the complement thereof.

The term “isolated” when used in reference to nucleic acids (e.g., genomic DNAs, cDNAs, mRNAs, or fragments thereof) is intended to mean that a nucleic acid molecule is present in a form that is substantially separated from other naturally occurring nucleic acids that are normally associated with the molecule. Specifically, since a naturally existing chromosome (or a viral equivalent thereof) includes a long nucleic acid sequence, an “isolated nucleic acid” as used herein means a nucleic acid molecule having only a portion of the nucleic acid sequence in the chromosome but not one or more other portions present on the same chromosome. More specifically, an “isolated nucleic acid” typically includes no more than 25 kb naturally occurring nucleic acid sequences which immediately flank the nucleic acid in the naturally existing chromosome (or a viral equivalent thereof). However, it is noted that an “isolated nucleic acid” as used herein is distinct from a clone in a conventional library such as genomic DNA library and cDNA library in that the clone in a library is still in admixture with almost all the other nucleic acids of a chromosome or cell. Thus, an “isolated nucleic acid” as used herein also should be substantially separated from other naturally occurring nucleic acids that are on a different chromosome of the same organism. Specifically, an “isolated nucleic acid” means a composition in which the specified nucleic acid molecule is significantly enriched so as to constitute at least 10% of the total nucleic acids in the composition.

An “isolated nucleic acid” can be a hybrid nucleic acid having the specified nucleic acid molecule covalently linked to one or more nucleic acid molecules that are not the nucleic acids naturally flanking the specified nucleic acid. For example, an isolated nucleic acid can be in a vector. In addition, the specified nucleic acid may have a nucleotide sequence that is identical to a naturally occurring nucleic acid or a modified form or mutein thereof having one or more mutations such as nucleotide substitution, deletion/insertion, inversion, and the like.

An isolated nucleic acid can be prepared from a recombinant host cell (in which the nucleic acids have been recombinantly amplified and/or expressed), or can be a chemically synthesized nucleic acid having a naturally occurring nucleotide sequence or an artificially modified form thereof.

The term “isolated polypeptide” as used herein is defined as a polypeptide molecule that is present in a form other than that found in nature. Thus, an isolated polypeptide can be a non-naturally occurring polypeptide. For example, an “isolated polypeptide” can be a “hybrid polypeptide.” An “isolated polypeptide” can also be a polypeptide derived from a naturally occurring polypeptide by additions or deletions or substitutions of amino acids. An isolated polypeptide can also be a “purified polypeptide” which is used herein to mean a composition or preparation in which the specified polypeptide molecule is significantly enriched so as to constitute at least 10% of the total protein content in the composition. A “purified polypeptide” can be obtained from natural or recombinant host cells by standard purification techniques, or by chemically synthesis, as will be apparent to skilled artisans.

As used herein, the term “BRCA nucleic acid” means a nucleic acid molecule the nucleotide sequence of which is uniquely found in a BRCA1 and/or BRCA2 gene. Thus, a “BRCA nucleic acid” is either a BRCA genomic DNA or mRNA/cDNA, having a naturally existing nucleotide sequence encoding a naturally existing BRCA protein (wild-type or mutant form). The sequence of an example of a naturally existing BRCA1 nucleic acid is found in GenBank Accession No. U14680. The sequence of an example of a naturally existing BRCA2 nucleic acid is found in GenBank Accession No. U43746. Both sequences can be found in the GenBank sequence database.

As used herein, the term “BRCA protein” means a polypeptide molecule the amino acid sequence of which is found uniquely in an BRCA protein (either BRCA1 and/or BRCA2). That is, “BRCA protein” is a naturally existing BRCA protein (wild-type or mutant form).

The term “locus” refers to a specific position or site in a gene sequence or protein. Thus, there may be one or more contiguous nucleotides in a particular gene locus, or one or more amino acids at a particular locus in a polypeptide. Moreover, “locus” may also be used to refer to a particular position in a gene where one or more nucleotides have been deleted, inserted, or inverted.

As used herein, the terms “polypeptide,” “protein,” and “peptide” are used interchangeably to refer to an amino acid chain in which the amino acid residues are linked by covalent peptide bonds. The amino acid chain can be of any length of at least two amino acids, including full-length proteins. Unless otherwise specified, the terms “polypeptide,” “protein,” and “peptide” also encompass various modified forms thereof, including but not limited to glycosylated forms, phosphorylated forms, etc.

The terms “primer”, “probe,” and “oligonucleotide” are used herein interchangeably to refer to a relatively short nucleic acid fragment or sequence. They can be DNA, RNA, or a hybrid thereof, or chemically modified analog or derivatives thereof. Typically, they are single-stranded. However, they can also be double-stranded having two complementing strands which can be separated apart by denaturation. In specific embodiments, the oligonucleotides can have a length of from about 8 nucleotides to about 200 nucleotides, or from about 12 nucleotides to about 100 nucleotides, or from about 18 to about 50 nucleotides. They can be labeled with detectable markers or modified in any conventional manners for various molecular biological applications.

The present invention also provides an isolated nucleic acid, naturally occurring or artificial, having a nucleotide sequence that is at least 50%, 60%, 70%, 75%, 80%, 85%, 90%, 95%, preferably at least 97% and more preferably at least 99% identical to one of SEQ ID NOs:3-32 except for containing one or more nucleotide variants of Table 1 and Table 2.

Also encompassed are isolated nucleic acids obtainable by:
(a) providing a human genomic library;
(b) screening the genomic library using a probe having a nucleotide sequence according to any one of SEQ ID NOs:1-32; and
(c) producing a genomic DNA comprising a contiguous span of at least 30 nucleotides of any one of SEQ ID NOs:3-32, wherein the genomic DNA thus produced contains one or more of the variants of the present invention in Table 1 and Table 2.

The present invention also includes isolated nucleic acids obtainable by:
(i) providing a cDNA library using human mRNA from a human tissue, e.g., blood;
(ii) screening the cDNA library using a probe having a nucleotide sequence according to any one of SEQ ID NOs:1-32; and
(iii) producing a cDNA DNA comprising a contiguous span of at least 30 nucleotides of any one of SEQ ID NOs:1-32, wherein the cDNA thus produced contains one or more of the nucleotide variants of the present invention in Table 1 and Table 2.

The present invention also encompasses an isolated nucleic acid comprising the nucleotide sequence of a region of a genomic DNA or cDNA or mRNA, wherein the region contains one or more nucleotide variants as provided in Table 1 and Table 2 above, or the complement thereof. Such regions can be isolated and analyzed to efficiently detect the nucleotide variants of the present invention. Also, such regions can also be isolated and used as probes or primers in detection of the nucleotide variants of the present invention and other uses as will be clear from the descriptions below.

Thus, in one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of a BRCA1 nucleic acid (e.g., SEQ ID NO:1), the contiguous span containing one or more nucleotide variants of Table 1 and Table 2, or the complement thereof. In specific embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of any human nucleic acid, said contiguous span containing one or more nucleotide variants of Table 1 and Table 2.

In one embodiment, the isolated nucleic acid comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:3-32, containing one or more nucleotide variants of Table 1, or the complement thereof. In specific embodiments, the isolated nucleic acid comprises a nucleotide sequence according to any one of SEQ ID NOs:3-32, or the complements thereof. In preferred embodiments, the isolated nucleic acids are oligonucleotides having a contiguous span of from about 17, 18, 19, 20, 21, 22, 23 or 25 to about 30, 40 or 50, preferably from about 21 to about 30 nucleotide residues, of any one of SEQ ID NOs:3-32 and containing one or more nucleotide variants selected from those in Table 1, or the complements thereof. The complements of the isolated nucleic acids are also encompassed by the present invention.

In preferred embodiments, an isolated oligonucleotide of the present invention is specific to an allele (“allele-specific”) containing one or more nucleotide variants as disclosed in the present invention, or the complement thereof. The term “allele” or “gene allele” is used herein to refer generally to a naturally occurring gene having a reference sequence or a gene containing a specific nucleotide variant. Thus, the isolated oligonucleotide may capable of selectively hybridizing, under high stringency conditions generally recognized in the art, to a genomic or cDNA or mRNA containing one or more nucleotide variants as disclosed in Table 1 and Table 2, but not to a genomic or cDNA or mRNA having an alternative nucleotide variant at the same locus or loci. Such oligonucleotides will be useful in a hybridization-based method for detecting the nucleotide variants of the present invention as described in details below. An ordinarily skilled artisan would recognize various stringent conditions which enable the oligonucleotides of the present invention to differentiate between different alleles at the same variant locus. For example, the hybridization can be conducted overnight in a solution containing 50% formamide, 5×SSC, pH7.6, 5×Denhardt's solution, 10% dextran sulfate, and 20 microgram/ml denatured, sheared salmon sperm DNA. The hybridization filters can be washed in 0.1×SSC at about 65° C. Alternatively, typical PCR conditions employed in the art with an annealing temperature of about 55° C. can also be used.

In the isolated oligonucleotides containing a nucleotide variant according to the present invention, the nucleotide variant (or the complement thereof) can be located in any position. In one embodiment, a nucleotide variant (or the complement thereof) is at the 5′ or 3′ end of the oligonucleotides. In a more preferred embodiment, an oligonucleotide contains only one nucleotide variant from Table 1 and Table 2 (or the complement thereof) according to the present invention, which is located at the 3′ end of the oligonucleotide. In another embodiment, a nucleotide variant (or the complement thereof) of the present invention is located within no greater than four (4), preferably no greater than three (3), and more preferably no greater than two (2) nucleotides of the center of the oligonucleotide of the present invention. In more preferred embodiment, a nucleotide variant (or the complement thereof) is located at the center or within one (1) nucleotide of the center of the oligonucleotide. For purposes of defining the location of a nucleotide variant in an oligonucleotide, the center nucleotide of an oligonucleotide with an odd number of nucleotides is considered to be the center. For an oligonucleotide with an even number of nucleotides, the bond between the two center nucleotides is considered to be the center.

In other embodiments of the present invention, isolated nucleic acids are provided which encode a contiguous span of at least 6, 7, 8, 9, 10, 11, 12, 13, 14, 15, 16 or 17 amino acids of a protein wherein said contiguous span contains at least one amino acid variant in Table 1 and Table 2 according to the present invention.

The oligonucleotides of the present invention can have a detectable marker selected from, e.g., radioisotopes, fluorescent compounds, enzymes, or enzyme co-factors operably linked to the oligonucleotide. The oligonucleotides of the present invention can be useful in genotyping as will be apparent from the description below.

In addition, the present invention also provides nucleic acid microchips or microarray incorporating one or more variant genomic DNA or cDNA or mRNA or an oligonucleotide according to the present invention. The microchips will allow rapid genotyping and/or haplotyping in a large scale efficiently. The microchips are also useful in determining quantitatively or qualitatively the expression of particularly variant alleles.

As is known in the art, in microchips, a large number of different nucleic acid probes are attached or immobilized in an array on a solid support, e.g., a silicon chip or glass slide. Target nucleic acid sequences to be analyzed can be contacted with the immobilized oligonucleotide probes on the microchip. See Lipshutz et al., Biotechniques, 19:442-447 (1995); Chee et al., Science, 274:610-614 (1996); Kozal et al., Nat. Med. 2:753-759 (1996); Hacia et al., Nat. Genet., 14:441-447 (1996); Saiki et al., Proc. Natl. Acad. Sci. USA, 86:6230-6234 (1989); Gingeras et al., Genome Res., 8:435-448 (1998). The microchip technologies combined with computerized analysis tools allow large-scale high throughput screening. See, e.g., U.S. Pat. No. 5,925,525 to Fodor et al; Wilgenbus et al., J. Mol. Med., 77:761-786 (1999); Graber et al., Curr. Opin. Biotechnol., 9:14-18 (1998); Hacia et al., Nat. Genet., 14:441-447 (1996); Shoemaker et al., Nat. Genet., 14:450-456 (1996); DeRisi et al., Nat. Genet., 14:457-460 (1996); Chee et al., Nat. Genet., 14:610-614 (1996); Lockhart et al., Nat. Genet., 14:675-680 (1996); Drobyshev et al., Gene, 188:45-52 (1997).

In a preferred embodiment, a DNA microchip is provided having a plurality of from 2 to 2000 oligonucleotides, or from 5 to 2000, or from 10 to 2000, or from 25 or 50 to 500, 1000, or 2000 oligonucleotides. In this preferred embodiment, each microchip includes at least 2, 3, 4, 5, 6, 7, 8, 9, 10, 12, 15, 20, 25, 30, 40 or 50, or at least 70, 80, 90 or 100 variant-containing oligonucleotides of the present invention each containing one different nucleotide variant selected from those in Table 1 and Table 2, or the complement thereof. In specific embodiments, each of the variant-containing oligonucleotides comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:3-32, and each contains one different nucleotide variant of those in Table 1, or the complement thereof. In preferred embodiments, each variant-containing oligonucleotide has a contiguous span of from about 17, 18, 19, 20, 21, 22, 23 or 25 to about 30, 40 or 50, preferably from about 21 to about 30, 40, 50 or 60 nucleotide residues, of any one of SEQ ID NOs:3-32, containing one nucleotide variant selected from those in Table 1, or the complement thereof.

The DNA microchip can be useful in detecting predisposition to DISEASE1, diagnosing DISEASE1, and selecting treatment or prevention regimens.

The terms “hybrid protein,” “hybrid polypeptide,” “hybrid peptide,” “fusion protein,” “fusion polypeptide,” and “fusion peptide” are used herein interchangeably to mean a non-naturally occurring polypeptide or isolated polypeptide having a specified polypeptide molecule covalently linked to one or more other polypeptide molecules that do not link to the specified polypeptide in nature. Thus, a “hybrid protein” may be two naturally occurring proteins or fragments thereof linked together by a covalent linkage. A “hybrid protein” may also be a protein formed by covalently linking two artificial polypeptides together. Typically but not necessarily, the two or more polypeptide molecules are linked or “fused” together by a peptide bond forming a single non-branched polypeptide chain.

The term “high stringency hybridization conditions,” when used in connection with nucleic acid hybridization, means hybridization conducted overnight at 42 degrees C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 0.1×SSC at about 65° C. The term “moderate stringency hybridization conditions,” when used in connection with nucleic acid hybridization, means hybridization conducted overnight at 37 degrees C in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

For the purpose of comparing two different nucleic acid or polypeptide sequences, one sequence (test sequence) may be described to be a specific “percentage identical to” another sequence (comparison sequence) in the present disclosure. In this respect, the percentage identity is determined by the algorithm of Karlin and Altschul, Proc. Natl. Acad. Sci. USA, 90:5873-5877 (1993), which is incorporated into various BLAST programs. Specifically, the percentage identity is determined by the “BLAST 2 Sequences” tool, which is available at NCBI's website. See Tatusova and Madden, FEMS Microbiol. Lett., 174(2):247-250 (1999). For pairwise DNA-DNA comparison, the BLASTN 2.1.2 program is used with default parameters (Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter). Percent identity of two sequences is calculated by aligning a test sequence with a comparison sequence using BLAST 2.1.2., determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence. When BLAST 2.1.2 is used to compare two sequences, it aligns the sequences and yields the percent identity over defined, aligned regions. If the two sequences are aligned across their entire length, the percent identity yielded by the BLAST 2.1.1 is the percent identity of the two sequences. If BLAST 2.1.2 does not align the two sequences over their entire length, then the number of identical amino acids or nucleotides in the unaligned regions of the test sequence and comparison sequence is considered to be zero and the percent identity is calculated by adding the number of identical amino acids or nucleotides in the aligned regions and dividing that number by the length of the comparison sequence.

The present invention also provides a method for genotyping by determining whether an individual has one or more of the nucleotide variants or amino acid variants of the present invention. The term “genotype” as used herein means the nucleotide characters at a particular nucleotide variant marker (or locus) in either one allele or both alleles of a gene (or a particular chromosome region). With respect to a particular nucleotide position of a gene of interest, the nucleotide(s) at that locus or equivalent thereof in one or both alleles form the genotype of the gene at that locus. A genotype can be homozygous or heterozygous. Accordingly, “genotyping” means determining the genotype, that is, the nucleotide(s) at a particular gene locus. Genotyping can also be done by determining the amino acid variant at a particular position of a protein which can be used to deduce the corresponding nucleotide variant(s). For purposes of genotyping and haplotyping, both genomic DNA and mRNA/cDNA can be used, and both are herein referred to generically as “gene.”

Numerous techniques for detecting nucleotide variants are known in the art and can all be used for the method of this invention. The techniques can be protein-based or DNA-based. In either case, the techniques used must be sufficiently sensitive so as to accurately detect the small nucleotide or amino acid variations. Very often, a probe is utilized which is labeled with a detectable marker. Unless otherwise specified in a particular technique described below, any suitable marker known in the art can be used, including but not limited to, radioactive isotopes, fluorescent compounds, biotin which is detectable using strepavidin, enzymes (e.g., alkaline phosphatase), substrates of an enzyme, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977).

In a DNA-based detection method, target DNA sample, i.e., a sample containing a genomic region of interest, or the corresponding cDNA or mRNA must be obtained from the individual to be tested. Any tissue or cell sample containing the relevant genomic DNA, mRNA, or cDNA or a portion thereof can be used. For this purpose, a tissue sample containing cell nucleus and thus genomic DNA can be obtained from the individual. Blood samples can also be useful except that only white blood cells and other lymphocytes have cell nucleus, while red blood cells are anucleate and contain only mRNA. Nevertheless, mRNA is also useful as it can be analyzed for the presence of nucleotide variants in its sequence or serve as template for cDNA synthesis. The tissue or cell samples can be analyzed directly without much processing. Alternatively, nucleic acids including the target sequence can be extracted, purified, and/or amplified before they are subject to the various detecting procedures discussed below. Other than tissue or cell samples, cDNAs or genomic DNAs from a cDNA or genomic DNA library constructed using a tissue or cell sample obtained from the individual to be tested are also useful.

To determine the presence or absence of a particular nucleotide variant, one technique is simply sequencing the target genomic DNA or cDNA, particularly the region encompassing the nucleotide variant locus to be detected. Various sequencing techniques are generally known and widely used in the art including the Sanger method and Gilbert chemical method. The newly developed pyrosequencing method monitors DNA synthesis in real time using a luminometric detection system. Pyrosequencing has been shown to be effective in analyzing genetic polymorphisms such as single-nucleotide polymorphisms and thus can also be used in the present invention. See Nordstrom et al., Biotechnol. Appl. Biochem., 31(2):107-112 (2000); Ahmadian et al., Anal. Biochem., 280:103-110 (2000).

Alternatively, the restriction fragment length polymorphism (RFLP) and AFLP method may also prove to be useful techniques. In particular, if a nucleotide variant in the target nucleic acid region results in the elimination or creation of a restriction enzyme recognition site, then digestion of the target DNA with that particular restriction enzyme will generate an altered restriction fragment length pattern. Thus, a detected RFLP or AFLP will indicate the presence of a particular nucleotide variant.

Another useful approach is the single-stranded conformation polymorphism assay (SSCA), which is based on the altered mobility of a single-stranded target DNA spanning the nucleotide variant of interest. A single nucleotide change in the target sequence can result in different intramolecular base pairing pattern, and thus different secondary structure of the single-stranded DNA, which can be detected in a non-denaturing gel. See Orita et al., Proc. Natl. Acad. Sci. USA, 86:2776-2770 (1989). Denaturing gel-based techniques such as clamped denaturing gel electrophoresis (CDGE) and denaturing gradient gel electrophoresis (DGGE) detect differences in migration rates of mutant sequences as compared to wild-type sequences in denaturing gel. See Miller et al., Biotechniques, 5:1016-24 (1999); Sheffield et al., Am. J. Hum, Genet., 49:699-706 (1991); Wartell et al., Nucleic Acids Res., 18:2699-2705 (1990); and Sheffield et al., Proc. Natl. Acad. Sci. USA, 86:232-236 (1989). In addition, the double-strand conformation analysis (DSCA) can also be useful in the present invention. See Arguello et al., Nat. Genet., 18:192-194 (1998).

The presence or absence of a nucleotide variant at a particular locus in a genomic region of an individual can also be detected using the amplification refractory mutation system (ARMS) technique. See e.g., European Patent No. 0,332,435; Newton et al., Nucleic Acids Res., 17:2503-2515 (1989); Fox et al., Br. J. Cancer, 77:1267-1274 (1998); Robertson et al., Eur. Respir. J., 12:477-482 (1998). In the ARMS method, a primer is synthesized matching the nucleotide sequence immediately 5′ upstream from the locus being tested except that the 3′-end nucleotide which corresponds to the nucleotide at the locus is a predetermined nucleotide. For example, the 3′-end nucleotide can be the same as that in the mutated locus. The primer can be of any suitable length so long as it hybridizes to the target DNA under stringent conditions only when its 3′-end nucleotide matches the nucleotide at the locus being tested. Preferably the primer has at least 12 nucleotides, more preferably from about 18 to 50 nucleotides. If the individual tested has a mutation at the locus and the nucleotide therein matches the 3′-end nucleotide of the primer, then the primer can be further extended upon hybridizing to the target DNA template, and the primer can initiate a PCR amplification reaction in conjunction with another suitable PCR primer. In contrast, if the nucleotide at the locus is of wild type, then primer extension cannot be achieved. Various forms of ARMS techniques developed in the past few years can be used. See e.g., Gibson et al., Clin. Chem. 43:1336-1341 (1997).

Similar to the ARMS technique is the mini sequencing or single nucleotide primer extension method, which is based on the incorporation of a single nucleotide. An oligonucleotide primer matching the nucleotide sequence immediately 5′ to the locus being tested is hybridized to the target DNA or mRNA in the presence of labeled dideoxyribonucleotides. A labeled nucleotide is incorporated or linked to the primer only when the dideoxyribonucleotides matches the nucleotide at the variant locus being detected. Thus, the identity of the nucleotide at the variant locus can be revealed based on the detection label attached to the incorporated dideoxyribonucleotides. See Syvanen et al., Genomics, 8:684-692 (1990); Shumaker et al., Hum. Mutat., 7:346-354 (1996); Chen et al., Genome Res., 10:549-547 (2000).

Another set of techniques useful in the present invention is the so-called “oligonucleotide ligation assay” (OLA) in which differentiation between a wild-type locus and a mutation is based on the ability of two oligonucleotides to anneal adjacent to each other on the target DNA molecule allowing the two oligonucleotides joined together by a DNA ligase. See Landergren et al., Science, 241:1077-1080 (1988); Chen et al, Genome Res., 8:549-556 (1998); Iannone et al., Cytometry, 39:131-140 (2000). Thus, for example, to detect a single-nucleotide mutation at a particular locus in a genomic region, two oligonucleotides can be synthesized, one having the genomic sequence just 5′ upstream from the locus with its 3′ end nucleotide being identical to the nucleotide in the variant locus, the other having a nucleotide sequence matching the genomic sequence immediately 3′ downstream from the variant locus. The oligonucleotides can be labeled for the purpose of detection. Upon hybridizing to the target nucleic acid under a stringent condition, the two oligonucleotides are subject to ligation in the presence of a suitable ligase. The ligation of the two oligonucleotides would indicate that the target DNA has a nucleotide variant at the locus being detected.

Detection of small genetic variations can also be accomplished by a variety of hybridization-based approaches. Allele-specific oligonucleotides are most useful. See Conner et al., Proc. Natl. Acad. Sci. USA, 80:278-282 (1983); Saiki et al, Proc. Natl. Acad. Sci. USA, 86:6230-6234 (1989). Oligonucleotide probes (allele-specific) hybridizing specifically to an allele having a particular nucleotide variant at a particular locus but not to other alleles can be designed by methods known in the art. The probes can have a length of, e.g., from 10 to about 50 nucleotide bases. The target DNA and the oligonucleotide probe can be contacted with each other under conditions sufficiently stringent such that the nucleotide variant can be distinguished from the alternative variant/allele at the same locus based on the presence or absence of hybridization. The probe can be labeled to provide detection signals. Alternatively, the allele-specific oligonucleotide probe can be used as a PCR amplification primer in an “allele-specific PCR” and the presence or absence of a PCR product of the expected length would indicate the presence or absence of a particular nucleotide variant.

Other useful hybridization-based techniques allow two single-stranded nucleic acids annealed together even in the presence of mismatch due to nucleotide substitution, insertion or deletion. The mismatch can then be detected using various techniques. For example, the annealed duplexes can be subject to electrophoresis. The mismatched duplexes can be detected based on their electrophoretic mobility that is different from the perfectly matched duplexes. See Cariello, Human Genetics, 42:726 (1988). Alternatively, in a RNase protection assay, a RNA probe can be prepared spanning the nucleotide variant site to be detected and having a detection marker. See Giunta et al., Diagn. Mol. Path., 5:265-270 (1996); Finkelstein et al., Genomics, 7:167-172 (1990); Kinszler et al., Science 251:1366-1370 (1991). The RNA probe can be hybridized to the target DNA or mRNA forming a heteroduplex that is then subject to the ribonuclease RNase A digestion. RNase A digests the RNA probe in the heteroduplex only at the site of mismatch. The digestion can be determined on a denaturing electrophoresis gel based on size variations. In addition, mismatches can also be detected by chemical cleavage methods known in the art. See e.g., Roberts et al., Nucleic Acids Res., 25:3377-3378 (1997).

In the mutS assay, a probe can be prepared matching the human nucleic acid sequence surrounding the locus at which the presence or absence of a nucleotide variant is to be detected, except that a predetermined nucleotide is used at the variant locus. Upon annealing the probe to the target DNA to form a duplex, the E. coli mutS protein is contacted with the duplex. Since the mutS protein binds only to heteroduplex sequences containing a nucleotide mismatch, the binding of the mutS protein will be indicative of the presence of a mutation. See Modrich et al., Ann. Rev. Genet., 25:229-253 (1991).

A great variety of improvements and variations have been developed in the art on the basis of the above-described basic techniques, and can all be useful in detecting mutations or nucleotide variants in the present invention. For example, the “sunrise probes” or “molecular beacons” utilize the fluorescence resonance energy transfer (FRET) property and give rise to high sensitivity. See Wolf et al., Proc. Nat. Acad. Sci. USA, 85:8790-8794 (1988). Typically, a probe spanning the nucleotide locus to be detected are designed into a hairpin-shaped structure and labeled with a quenching fluorophore at one end and a reporter fluorophore at the other end. In its natural state, the fluorescence from the reporter fluorophore is quenched by the quenching fluorophore due to the proximity of one fluorophore to the other. Upon hybridization of the probe to the target DNA, the 5′ end is separated apart from the 3′-end and thus fluorescence signal is regenerated. See Nazarenko et al., Nucleic Acids Res., 25:2516-2521 (1997); Rychlik et al., Nucleic Acids Res., 17:8543-8551 (1989); Sharkey et al., Bio/Technology 12:506-509 (1994); Tyagi et al., Nat. Biotechnol., 14:303-308 (1996); Tyagi et al., Nat. Biotechnol., 16:49-53 (1998). The homo-tag assisted non-dimer system (HANDS) can be used in combination with the molecular beacon methods to suppress primer-dimer accumulation. See Brownie et al., Nucleic Acids Res., 25:3235-3241 (1997).

Dye-labeled oligonucleotide ligation assay is a FRET-based method, which combines the OLA assay and PCR. See Chen et al., Genome Res. 8:549-556 (1998). TaqMan is another FRET-based method for detecting nucleotide variants. A TaqMan probe can be oligonucleotides designed to have the nucleotide sequence of the human nucleic acid spanning the variant locus of interest and to differentially hybridize with different alleles. The two ends of the probe are labeled with a quenching fluorophore and a reporter fluorophore, respectively. The TaqMan probe is incorporated into a PCR reaction for the amplification of a target nucleic acid region containing the locus of interest using Taq polymerase. As Taq polymerase exhibits 5′-3′ exonuclease activity but has no 3′-5′ exonuclease activity, if the TaqMan probe is annealed to the target DNA template, the 5′-end of the TaqMan probe will be degraded by Taq polymerase during the PCR reaction thus separating the reporting fluorophore from the quenching fluorophore and releasing fluorescence signals. See Holland et al., Proc. Natl. Acad. Sci. USA, 88:7276-7280 (1991); Kalinina et al., Nucleic Acids Res., 25:1999-2004 (1997); Whitcombe et al., Clin. Chem., 44:918-923 (1998).

In addition, the detection in the present invention can also employ a chemiluminescence-based technique. For example, an oligonucleotide probe can be designed to hybridize to either the wild-type or a variant locus but not both. The probe is labeled with a highly chemiluminescent acridinium ester. Hydrolysis of the acridinium ester destroys chemiluminescence. The hybridization of the probe to the target DNA prevents the hydrolysis of the acridinium ester. Therefore, the presence or absence of a particular mutation in the target DNA is determined by measuring chemiluminescence changes. See Nelson et al., Nucleic Acids Res., 24:4998-5003 (1996).

The detection of genetic variation in accordance with the present invention can also be based on the “base excision sequence scanning” (BESS) technique. The BESS method is a PCR-based mutation scanning method. BESS T-Scan and BESS G-Tracker are generated which are analogous to T and G ladders of dideoxy sequencing. Mutations are detected by comparing the sequence of normal and mutant DNA. See, e.g., Hawkins et al., Electrophoresis, 20:1171-1176 (1999).

Another useful technique that is gaining increased popularity is mass spectrometry. See Graber et al., Curr. Opin. Biotechnol., 9:14-18 (1998). For example, in the primer oligo base extension (PROBE™) method, a target nucleic acid is immobilized to a solid-phase support. A primer is annealed to the target immediately 5′ upstream from the locus to be analyzed. Primer extension is carried out in the presence of a selected mixture of deoxyribonucleotides and dideoxyribonucleotides. The resulting mixture of newly extended primers is then analyzed by MALDI-TOF. See e.g., Monforte et al., Nat. Med., 3:360-362 (1997).

In addition, the microchip or microarray technologies are also applicable to the detection method of the present invention as will be apparent to a skilled artisan in view of this disclosure. For example, to genotype an individual, genomic DNA isolated from the individual can be prepared and hybridized to a DNA microchip of the present invention as described above in Section 3, and the genotypes at a plurality of loci can be determined.

As is apparent from the above survey of the suitable detection techniques, it may or may not be necessary to amplify the target DNA, i.e., the genomic region of interest, or the corresponding cDNA or mRNA to increase the number of target DNA molecule, depending on the detection techniques used. For example, most PCR-based techniques combine the amplification of a portion of the target and the detection of the mutations. PCR amplification is well known in the art and is disclosed in U.S. Pat. Nos. 4,683,195 and 4,800,159, both which are incorporated herein by reference. For non-PCR-based detection techniques, if necessary, the amplification can be achieved by, e.g., in vivo plasmid multiplication, or by purifying the target DNA from a large amount of tissue or cell samples. See generally, Sambrook et al., Molecular Cloning: A Laboratory Manual, 2nd ed., Cold Spring Harbor Laboratory, Cold Spring Harbor, N.Y., 1989. However, even with scarce samples, many sensitive techniques have been developed in which small genetic variations such as single-nucleotide substitutions can be detected without having to amplify the target DNA in the sample. For example, techniques have been developed that amplify the signal as opposed to the target DNA by, e.g., employing branched DNA or dendrimers that can hybridize to the target DNA. The branched or dendrimer DNAs provide multiple hybridization sites for hybridization probes to attach thereto thus amplifying the detection signals. See Detmer et al., J. Clin. Microbiol., 34:901-907 (1996); Collins et al., Nucleic Acids Res., 25:2979-2984 (1997); Horn et al., Nucleic Acids Res., 25:4835-4841 (1997); Horn et al., Nucleic Acids Res., 25:4842-4849 (1997); Nilsen et al., J. Theor. Biol., 187:273-284 (1997).

In yet another technique for detecting single nucleotide variations, the Invader® assay utilizes a novel linear signal amplification technology that improves upon the long turnaround times required of the typical PCR DNA sequenced-based analysis. See Cooksey et al., Antimicrobial Agents and Chemotherapy 44:1296-1301 (2000). This assay is based on cleavage of a unique secondary structure formed between two overlapping oligonucleotides that hybridize to the target sequence of interest to form a “flap.” Each “flap” then generates thousands of signals per hour. Thus, the results of this technique can be easily read, and the methods do not require exponential amplification of the DNA target. The Invader® system utilizes two short DNA probes, which are hybridized to a DNA target. The structure formed by the hybridization event is recognized by a special cleavase enzyme that cuts one of the probes to release a short DNA “flap.” Each released “flap” then binds to a fluorescently-labeled probe to form another cleavage structure. When the cleavase enzyme cuts the labeled probe, the probe emits a detectable fluorescence signal. See e.g. Lyamichev et al., Nat. Biotechnol., 17:292-296 (1999).

The rolling circle method is another method that avoids exponential amplification. Lizardi et al., Nature Genetics, 19:225-232 (1998) (which is incorporated herein by reference). For example, Sniper™, a commercial embodiment of this method, is a sensitive, high-throughput SNP scoring system designed for the accurate fluorescent detection of specific variants. For each nucleotide variant, two linear, allele-specific probes are designed. The two allele-specific probes are identical with the exception of the 3′-base, which is varied to complement the variant site. In the first stage of the assay, target DNA is denatured and then hybridized with a pair of single, allele-specific, open-circle oligonucleotide probes. When the 3′-base exactly complements the target DNA, ligation of the probe will preferentially occur. Subsequent detection of the circularized oligonucleotide probes is by rolling circle amplification, whereupon the amplified probe products are detected by fluorescence. See Clark and Pickering, Life Science News 6, 2000, Amersham Pharmacia Biotech (2000).

A number of other techniques that avoid amplification all together include, e.g., surface-enhanced resonance Raman scattering (SERRS), fluorescence correlation spectroscopy, and single-molecule electrophoresis. In SERRS, a chromophore-nucleic acid conjugate is absorbed onto colloidal silver and is irradiated with laser light at a resonant frequency of the chromophore. See Graham et al., Anal. Chem., 69:4703-4707 (1997). The fluorescence correlation spectroscopy is based on the spatio-temporal correlations among fluctuating light signals and trapping single molecules in an electric field. See Eigen et al., Proc. Natl. Acad. Sci. USA, 91:5740-5747 (1994). In single-molecule electrophoresis, the electrophoretic velocity of a fluorescently tagged nucleic acid is determined by measuring the time required for the molecule to travel a predetermined distance between two laser beams. See Castro et al., Anal. Chem., 67:3181-3186 (1995).

In addition, the allele-specific oligonucleotides (ASO) can also be used in in situ hybridization using tissues or cells as samples. The oligonucleotide probes which can hybridize differentially with the wild-type gene sequence or the gene sequence harboring a mutation may be labeled with radioactive isotopes, fluorescence, or other detectable markers. In situ hybridization techniques are well known in the art and their adaptation to the present invention for detecting the presence or absence of a nucleotide variant in a genomic region of a particular individual should be apparent to a skilled artisan apprised of this disclosure.

Protein-based detection techniques may also prove to be useful, especially when the nucleotide variant causes amino acid substitutions or deletions or insertions or frameshift that affect the protein primary, secondary or tertiary structure. To detect the amino acid variations, protein sequencing techniques may be used. For example, a protein or fragment thereof can be synthesized by recombinant expression using an encoding cDNA fragment isolated from an individual to be tested. Preferably, a cDNA fragment of no more than 100 to 150 base pairs encompassing the polymorphic locus to be determined is used. The amino acid sequence of the peptide can then be determined by conventional protein sequencing methods. Alternatively, the recently developed HPLC-microscopy tandem mass spectrometry technique can be used for determining the amino acid sequence variations. In this technique, proteolytic digestion is performed on a protein, and the resulting peptide mixture is separated by reversed-phase chromatographic separation. Tandem mass spectrometry is then performed and the data collected therefrom is analyzed. See Gatlin et al., Anal. Chem., 72:757-763 (2000).

Other useful protein-based detection techniques include immunoaffinity assays based on antibodies selectively immunoreactive with mutant proteins according to the present invention. The method for producing such antibodies is described above in detail. Antibodies can be used to immunoprecipitate specific proteins from solution samples or to immunoblot proteins separated by, e.g., polyacrylamide gels. Immunocytochemical methods can also be used in detecting specific protein polymorphisms in tissues or cells. Other well-known antibody-based techniques can also be used including, e.g., enzyme-linked immunosorbent assay (ELISA), radioimmunoassay (RIA), immunoradiometric assays (IRMA) and immunoenzymatic assays (IEMA), including sandwich assays using monoclonal or polyclonal antibodies. See e.g., U.S. Pat. Nos. 4,376,110 and 4,486,530, both of which are incorporated herein by reference.

Accordingly, the presence or absence of a nucleotide variant or amino acid variant in an individual can be determined using any of the detection methods described above.

The present invention also provides a kit for genotyping, i.e., determining the presence or absence of one or more of the nucleotide or amino acid variants of present invention in the genomic DNA, or cDNA or mRNA in a sample obtained from a patient. The kit may include a carrier for the various components of the kit. The carrier can be a container or support, in the form of, e.g., bag, box, tube, rack, and is optionally compartmentalized. The carrier may define an enclosed confinement for safety purposes during shipment and storage. The kit also includes various components useful in detecting nucleotide or amino acid variants discovered in accordance with the present invention using the above-discussed detection techniques.

In one embodiment, the detection kit includes one or more oligonucleotides useful in detecting one or more of the nucleotide variants in Table 1. The oligonucleotides can be in one or more compartments or containers in the kit. In a preferred embodiment, the kit has a plurality of from 2 to 2000 oligonucleotides, or from 5 to 2000, or from 10 to 2000, or from 25 or 50 to 500, 1000, 1500 or 2000 oligonucleotides. In this preferred embodiment, each kit includes at least 2, 3, 4, 5, 6, 7, 8, 9, 10, 12, 15, 20, 25, 30, 40 or 50, or at least 70, 80, 90 or 100 variant-containing oligonucleotides of the present invention each containing one different nucleotide variant selected from those in Table 1 and Table 2, or the complement thereof. In specific embodiments, each of the variant-containing oligonucleotides comprises a contiguous span of at least 12, 15, 17, 18, 19, 20, 21, 22, 23, 24, 25, 26, 27, 28, 29, 30, 40, 50, 70 or 100 nucleotide residues of any one of SEQ ID NOs:3-32, and each contains one different nucleotide variant of those in Table 1, or the complement thereof. In preferred embodiments, each variant-containing oligonucleotide has a contiguous span of from about 17, 18, 19, 20, 21, 22, 23 or 25 to about 30, 40 or 50, preferably from about 21 to about 30, 40, 50 or 60 nucleotide residues, of any one of SEQ ID NOs:3-32, containing one nucleotide variant selected from those in Table 1, or the complement thereof.

In the kit of the present invention having oligonucleotides, the oligonucleotides can be affixed to a solid support, e.g., incorporated in a microchip or microarray included in the kit. In other words, microchips and microarrays according to the present invention described above in Section 3 can be included in the kit.

Preferably, the oligonucleotides are allele-specific, i.e., are designed such that they hybridize only to a human nucleic acid of a particular allele, i.e., containing a particular nucleotide variant (versus the alternative variant at the same locus) discovered in accordance with the present invention, under stringent conditions. Thus, the oligonucleotides can be used in mutation-detecting techniques such as allele-specific oligonucleotides (ASO), allele-specific PCR, TaqMan, chemiluminescence-based techniques, molecular beacons, and improvements or derivatives thereof, e.g., microchip technologies. The oligonucleotides in this embodiment preferably have a nucleotide sequence that matches a nucleotide sequence of a variant allele containing a nucleotide variant to be detected. The length of the oligonucleotides in accordance with this embodiment of the invention can vary depending on its nucleotide sequence and the hybridization conditions employed in the detection procedure. Preferably, the oligonucleotides contain from about 10 nucleotides to about 100 nucleotides, more preferably from about 15 to about 75 nucleotides, e.g., a contiguous span of 18, 19, 20, 21, 22, 23, 24 or 25 to 21, 22, 23, 24, 26, 27, 28, 29 or 30 nucleotide residues of a nucleic acid one or more of the residues being a nucleotide variant of the present invention, i.e., selected from Table 1 and Table 2. Under some conditions, a length of 18 to 30 may be optimum. In any event, the oligonucleotides should be designed such that it can be used in distinguishing one nucleotide variant from another at a particular locus under predetermined stringent hybridization conditions. Preferably, a nucleotide variant is located at the center or within one (1) nucleotide of the center of the oligonucleotides, or at the 3′ or 5′ end of the oligonucleotides. The hybridization of an oligonucleotide with a nucleic acid and the optimization of the length and hybridization conditions should be apparent to a person of skill in the art. See generally, Sambrook et al., Molecular Cloning: A Laboratory Manual, 2nd ed., Cold Spring Harbor Laboratory, Cold Spring Harbor, N.Y., 1989. Notably, the oligonucleotides in accordance with this embodiment are also useful in mismatch-based detection techniques described above, such as electrophoretic mobility shift assay, RNase protection assay, mutS assay, etc.

In another embodiment of this invention, the kit includes one or more oligonucleotides suitable for use in detecting techniques such as ARMS, oligonucleotide ligation assay (OLA), and the like. The oligonucleotides in this embodiment include a human nucleic acid sequence of about 10 to about 100 nucleotides, preferably from about 15 to about 75 nucleotides, e.g., contiguous span of 18, 19, 20, 21, 22, 23, 24 or 25 to 21, 22, 23, 24, 26, 27, 28, 29 or 30 nucleotide residues immediately 5′ upstream from the nucleotide variant to be analyzed. The 3′ end nucleotide in such oligonucleotides is a nucleotide variant in accordance with this invention.

The oligonucleotides in the detection kit can be labeled with any suitable detection marker including but not limited to, radioactive isotopes, fluorephores, biotin, enzymes (e.g., alkaline phosphatase), enzyme substrates, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977). Alternatively, the oligonucleotides included in the kit are not labeled, and instead, one or more markers are provided in the kit so that users may label the oligonucleotides at the time of use.

In another embodiment of the invention, the detection kit contains one or more antibodies selectively immunoreactive with certain protein variants containing specific amino acid variants discovered in the present invention. Methods for producing and using such antibodies have been described above in detail.

Various other components useful in the detection techniques may also be included in the detection kit of this invention. Examples of such components include, but are not limited to, Taq polymerase, deoxyribonucleotides, dideoxyribonucleotides other primers suitable for the amplification of a target DNA sequence, RNase A, mutS protein, and the like. In addition, the detection kit preferably includes instructions on using the kit for detecting nucleotide variants in human samples.

### Example 1

The purpose of this study was to investigate the incidence of germline and somatic BRCA1/2 deleterious mutations in an unselected group of patients with TNBC, and to determine the prognostic significance of carrying a mutation by assessing relapse-free (RFS) and overall survival (OS).

### Methods

Patients and Treatment

As part of a TNBC molecular characterization project the Breast Cancer Management System Database at The University of Texas M.D. Anderson Cancer Center (MDACC) was searched to identify patients with invasive TNBC who had definitive surgery and from whom tumor and normal tissue was available from the MDACC Breast Cancer Tumor Bank. Ninety-six primary frozen tumors were identified. Normal tissues were available in 77 cases diagnosed between 1997 and 2006. No germline DNA was extracted from blood. All specimens and clinical information were collected under Institutional Review Board (IRB)-approved protocols.

Pathology and Mutation Analysis

Dedicated breast pathologists at MDACC reviewed all pathologic specimens. Diagnosis of invasive TNBC cancer was made by core-needle biopsy of the breast tumor. Clinical stage was defined by the sixth edition of the American Joint Committee on Cancer (AJCC) Cancer Staging Manual. The histologic type of all tumors was defined according to the World Health Organization's classification system. Tumor grade was defined according to the modified Black's nuclear grading system. TNBC was define as negative ER, PR and HER2 status. Immunohistochemical analysis to determine ER and PR status was performed using standard immunohistochemistry (IHC) procedures with monoclonal antibodies. Nuclear staining less than or equal to 5% was considered a negative result. HER2 status was evaluated by IHC or by fluorescence in situ hybridization (FISH). HER2-negative tumors were defined as 0 or 1+ receptor over-expression on IHC staining and/or lack of gene amplification found on FISH testing (ratio equal or greater than 2.0).

DNA Extraction from frozen tissues was performed using sections in Tissue-Tek OCT (QIAgen, Valencia, Calif.) which were homogenized using a TissueRuptor (QIAgen, Valencia, Calif.) after adding QIAzol lysis reagent. A QIAamp DNA MiniKit (QIAgen, Valencia, Calif.) was used to isolate DNA per manufacturer's protocol with overnight incubation (56° C.) and RNaseA treatment.

For mutation screening, PCR was performed on 2 ng DNA in a 3 uL reaction using the primers flanking the exons of BRCA1/BRCA2 that are used in the BRACAnalysis® (Myriad Genetics, Salt Lake City, Utah) clinical test with the following cycling conditions: 95° C.×10 minutes, 35 cycles of 95° C.×30 seconds, 62° C.×30 seconds and 72° C.×1 minute, finishing with 72° C.×1 minute. Each PCR product was treated with 0.1 U Shrimp Alkaline Phosphatase (Sigma-Aldrich Inc.) The PCR product was diluted 1:9 and 0.8 uL was used for cycle sequencing with Big Dye Sequencing Chemistry and Taq FS (Applied Biosystems). Cycle conditions were 95° C.×3 minutes, 32 cycles of 95° C.×30 seconds, 50° C.×30 seconds, 60° C.×3 minutes, 72° C.×10 minutes. Sequence products were run on a Megabace 4500 automated sequencer (GE) per manufacturer's protocol.

BRCA1/BRCA2 mutations were only included in the analyses below if classified as deleterious or suspected deleterious based on established criteria. In patients in whom BRCA1/BRCA2 mutations were identified, germline DNA (from blood or normal breast) was used to test for BRCA1/BRCA2 mutations. Patients with mutations in tumor and normal tissue were classified as having germline mutations, patients with mutations in the tumor but not normal tissue were classified as having somatic mutations.

Statistical Methods

Patient characteristics have been tabulated and described by their medians or ranges, and compared between groups (mutation carriers vs. wild type) by a chi-square test or Wilcoxon's rank sum test, as appropriate. Time to recurrence was measured from the date of diagnosis to the date of local or systemic recurrence or the last follow-up. Patients who died before experiencing a disease recurrence were considered censored at their date of death in the analysis of recurrence-free survival (RFS). Survival time was measured from the date of diagnosis to the date of death, or the last follow-up. Median survival time was calculated as the median observation time among all patients.

Survival outcomes were estimated according to the Kaplan-Meier product limit method, and compared between groups by the log-rank statistic. Cox proportional hazard model was employed to determine the association of breast cancer subtype with the risk of recurrence after adjustment for other significant patient and disease characteristics. All terms that were significantly associated with recurrence-free survival (i.e. P-value<0.05) were considered and included in a multivariable model. Final model was based on either statistical or clinical significance. All analyses were performed using R 2.10.1 (R Development Core Team http://www.R-project.org).

### Results

The patients' characteristics are summarized in Table 3. Median age was 51 years (range 27-83 years). Of the 77 patients identified, 15 (19.5%) had BRCA mutations: 12 (15.6%) in BRCA1, one of them somatic, and 3 (3.9%) in BRCA2. Table 2 describes the complete list of deleterious mutations found in the cohort. Compared to wild type, patients with BRCA mutations tended to be younger, (p=0.005). Nuclear grade, histology and pathology stage were not significantly associated with mutation status.

TABLE 3
Patient and Tumor Characteristics

| | | | BRCA | | BRCA Wild- | | |
|---|---|---|---|---|---|---|---|
| | Total | | Mutant | | Type | | |
| Characteristic | N | % | N | % | N | % | p-value |
| | 77 | 100% | 15 | 19.50% | 62 | 80.50% | — |
| Age at | | | | | | | |
| Diagnosis | | | | | | | |
| Median | 51 | (27-83) | 45 | (27-61) | 53 | (28-83) | 0.0051 |
| Race | | | | | | | |
| Black | 18 | 23.37% | 2 | 13.33% | 16 | 25.81% | |
| Hispanic | 8 | 10.38% | 3 | 20.00% | 5 | 8.06% | |
| White | 50 | 64.93% | 10 | 66.67% | 40 | 64.52% | |
| Other | 1 | 1.29% | 0 | 0.00% | 1 | 1.61% | 0.4385 |
| Menopausal | | | | | | | |
| Status | | | | | | | |
| Pre- | 29 | 37.66% | 8 | 53.33% | 21 | 33.87% | |
| menopausal | | | | | | | |
| Post- | 48 | 62.33% | 7 | 46.67% | 41 | 66.13% | 0.2718 |
| menopausal | | | | | | | |
| Histology | | | | | | | |
| Ductal | 64 | 83.11% | 12 | 80.00% | 52 | 83.87% | |
| Other | 13 | 16.88% | 3 | 20.00% | 10 | 16.13% | 0.7096 |
| Pathological | | | | | | | |
| Stage | | | | | | | |
| I and II | 47 | 61.03% | 8 | 53.33% | 39 | 62.90% | |
| III | 30 | 38.96% | 7 | 46.67% | 23 | 37.10% | 0.6988 |
| Nuclear Grade | | | | | | | |
| 2 | 5 | 6.49% | 2 | 13.33% | 3 | 4.84% | |
| 3 | 70 | 90.90% | 13 | 86.67% | 57 | 91.94% | 0.2598 |
| Lympho- | | | | | | | |
| vascular | | | | | | | |
| Invasion | | | | | | | |
| Positive | 18 | 23.37% | 3 | 20.00% | 15 | 24.19% | |
| Negative | 57 | 74.02% | 12 | 80.00% | 45 | 72.58% | 1 |

In general, from all 77 patients, 33 (43%) were referred to genetic counseling for evaluation. Twenty-two patients (30%) had a positive family history for breast and/or ovarian cancer. Twelve of these patients had at least one first degree family member diagnosed with either malignancy. From these 22 patients, twelve were referred to genetic evaluation, and eight were tested, five of whom tested positive for a deleterious mutation in BRCA1. From the 33 patients referred to genetic counseling, genetic testing was recommended on 28, and completed on 17. Eleven patients declined testing, one patient declined counseling.

Six out of the 14 germline mutation carriers and the patient who has the tumor with the somatic BRCA1 mutation were not referred to genetic counseling. Nine out of the 14 germline mutation carriers had no first degree family history of breast and/or ovarian cancer. Two patients refused testing, and testing was not recommended in two as they did not meet standard guidelines for testing.

Twenty five (37.8%) patients were treated with breast-conserving treatment, nine with unilateral mastectomy (one with a planned delayed contra lateral risk-reducing mastectomy). Nine patients had a bilateral mastectomy, four for bilateral breast cancer and five for contralateral risk reduction. From the 14 patients with germline mutations, four had a bilateral mastectomy, two of them for bilateral breast cancer, and one had a planned delayed contra lateral risk-reducing mastectomy. Two patients declined further surgery, and one patient declined genetic counseling or further surgery. One developed metastatic ovarian cancer during breast cancer therapy. Three patients had unilateral mastectomy and two had breast conserving surgery.

All 25 patients who underwent breast-conserving surgery received adjuvant radiation therapy. Sixteen (40%) of 40 WT patients and six (50%) of twelve patients with BRCA mutations who underwent a mastectomy received adjuvant radiation therapy.

All patients but one received adjuvant chemotherapy. Adjuvant chemotherapy consisted of FAC (5 fluorouracil 500 mg/m² intravenously (IV) on days 1 and 4, doxorubicin 50 mg/m² IV continuous infusion over 72 hours and cyclophosphamide 500 mg/m² IV on day 1, every 3 weeks) for 4 to 6 courses (1 patient), FEC (5 fluorouracil 500 mg/m² IV, epirubicin 100 mg/m² IV, and cyclophosphamide 500 mg/m² IV on day 1, every 3 weeks) for 4 cycles and taxane (paclitaxel 175-250 mg/m², or docetaxel 100 mg/m² every 21 days for 4 cycles, or paclitaxel 80 mg/m² weekly for 12 weeks).

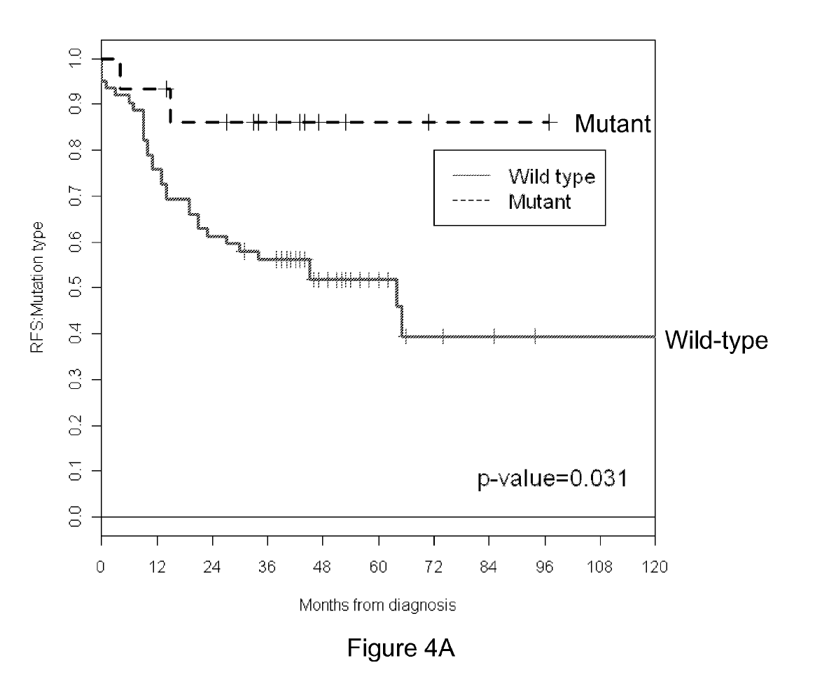

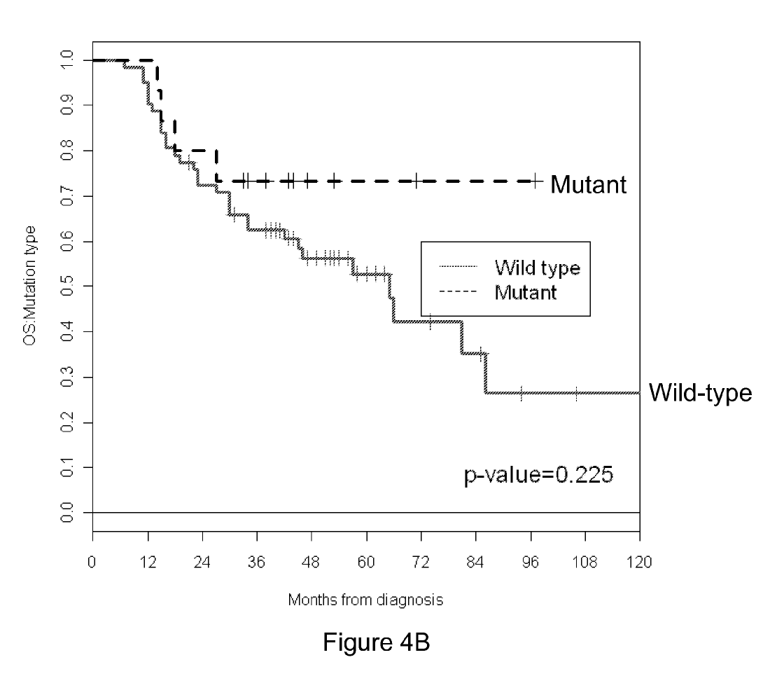

At a median follow-up of 43 months (range 7-214 months), there were 33 (42.9%) recurrences and 35 (45.5%) deaths. Three patients died without relapse and only one patient who has relapsed is still alive. Survival estimates are summarized in Table 4. Five-year RFS estimates were 51.7% for wild type patients vs. 86.2% for patients with BRCA mutations, (p=0.031); and 5-year OS estimates were 52.8% for wild type patients vs. 73.3% for or patients with BRCA mutations, (p=0.225). The Kaplan Meier plots for RFS and OS by mutational status are shown in FIG. 4 (A and B).

TABLE 4
Survival Estimates

| | | | | |
|---|---|---|---|---|
| N at | N | 5-Year | 95% Confidence | P- |
| risk | Events | Estimate | Interval | Value |

Recurrence-Free Survival Estimates

| | | | | | |
|---|---|---|---|---|---|
| All | | 33 | 57.80% | (47.3%, 0.6%) | |
| Race | | | | | |
| Black | 18 | 10 | 38.70% | (20.4, 73.3%) | |
| Hispanic | 8 | 2 | 75.00% | (50.3%, 100%) | |
| White | 50 | 20 | 63.60% | (51.5, 78.6%) | |
| Other | 1 | 1 | — | — | 0.237 |
| Age | | | | | |
| ≦50 | 36 | 13 | 65.30% | (50.2%, 85.0%) | |
| >50 | 41 | 20 | 51.20% | (38.0%, 69.1%) | 0.121 |
| Menopausal | | | | | |
| Pre- | 29 | 12 | 62.80% | (46.5%, 84.7%) | |
| menopausal | | | | | |
| Post- | 48 | 21 | 54.60% | (41.7%, 71.5%) | 0.553 |
| menopausal | | | | | |
| Histology | | | | | |
| Ductal | 64 | 28 | 57.70% | (46.4%, 71.8%) | |
| Other | 13 | 5 | 59.80% | (37.8%, 94.7%) | 0.951 |
| Pathological | | | | | |
| I and II | 47 | 13 | 74.10% | (61.7%, 89.0%) | |
| III | 30 | 20 | 32.30% | (19.1%, 54.7%) | <0.001. |
| Nuclear Grade | | | | | |
| 2 | 5 | 1 | 80.00% | (51.6%, 100%) | |
| 3 | 70 | 32 | 54.90% | (43.9%, 68.8%) | 0.264 |
| Lympho- | | | | | |
| vascular | | | | | |
| Invasion | | | | | |
| Positive | 18 | 10 | 44.40% | (26.5%, 74.5%) | |
| Negative | 57 | 22 | 62.30% | (50.1%, 77.3%) | 0.073 |
| Mutation Status | | | | | |
| Mutant | 15 | 2 | 86.20% | (70.0%, 100%) | |
| Wild Type | 62 | 31 | 51.70% | (40.3%, 66.7%) | 0.031 |

Overall Survival Estimates

| | | | | | |
|---|---|---|---|---|---|
| All | | 35 | 55.90% | (44.7%, 70.0%) | |
| Race | | | | | |
| Black | 18 | 10 | 30.80% | (11.8%, 80.5%) | |
| Spanish/ | 8 | 2 | 75.00% | (50.3%, 100%) | |
| Hispanic | | | | | |
| White | 50 | 22 | 62.70% | (50.2%, 78.2%) | |
| Other | 1 | 1 | — | — | 0.006 |
| Age | | | | | |
| ≦50 | 36 | 15 | 60.70% | (44.6%, 82.7%) | |
| >50 | 41 | 20 | 53.40% | (40.1%, 71.2%) | 0.279 |
| Menopausal | | | | | |
| Pre- | 29 | 13 | 59.70% | (42.0%, 84.9%) | |
| menopausal | | | | | |
| Post- | 48 | 22 | 54.80% | (41.9%, 71.6%) | 0.626 |
| menopausal | | | | | |
| Histology | | | | | |
| Ductal | 64 | 29 | 57.00% | (44.8%, 72.6%) | |
| Other | 13 | 6 | 51.30% | (29.6%, 88.8%) | 0.681 |
| Pathological | | | | | |
| I and II | 47 | 14 | 73.30% | (59.4%, 90.6%) | |
| III | 30 | 21 | 29.60% | (17.0%, 51.7%) | <0.001. |
| Nuclear Grade | | | | | |
| 2 | 5 | 2 | 80.00% | (51.6%, 100%) | |
| 3 | 70 | 33 | 52.80% | (41.0%, 68.1%) | 0.675 |
| Lympho- | | | | | |
| vascular | | | | | |
| invasion | | | | | |
| Positive | 18 | 10 | 44.40% | (26.5%, 74.5%) | |
| Negative | 57 | 24 | 59.50% | (46.2%, 76.7%) | 0.246 |
| Mutation Status | | | | | |
| Mutant | 15 | 4 | 73.30% | (54.0%, 99.5%) | |
| Wild Type | 62 | 31 | 52.80% | (40.7%, 68.5%) | 0.225 |

Table 5 summarizes the results of the multivariable models for RFS and OS. After adjustment for other patient characteristics, patients with BRCA mutations had a significantly better RFS(HR: 0.19, 95% CI: 0.045-0.79, p=0.016) compared to no mutation carriers.

TABLE 5
Multivariable Cox Proportional Hazards Models

| | | |
|---|---|---|
| Hazard | 95% Confidence | |
| Ratio | Interval | P-value |

Recurrence-Free Survival Estimates

| | | | |
|---|---|---|---|
| Mutation | | | |
| Status | | | |
| Wild Type | 1 | | |
| Mutant | 0.17 | (0.041, 0.72) | 0.016 |
| Pathological | | | |
| Stage | | | |
| I and II | 1 | | |
| III | 5.32 | (2.55, 11.10) | <0.0001 |
| Nuclear Grade | | | |
| 2 | 1 | | |
| 3 | 1.25 | (0.16, 9.65) | 0.83 |

Overall Survival

| | | | |
|---|---|---|---|
| Mutation | | | |
| Status | | | |
| Wild Type | 1 | | |
| Mutant | 0.45 | (0.16, 1.29) | 0.138 |
| Pathological | | | |
| Stage | | | |
| I and II | 1 | | |
| III | 4.44 | (2.16, 9.12) | <0.0001 |
| Nuclear Grade | | | |
| 2 | 1 | | |
| 3 | 0.63 | (0.14, 2.85) | 0.55 |

### Discussion

In this unselected cohort of patients with TNBC, we found a 19.5% incidence of BRCA mutations. The frequency of somatic and germline BRCA mutations in unselected TNBC has not been described before. In this unselected cohort of patients with TNBC, we found a 19.5% incidence of BRCA mutations. From all 77 patients, 35 were referred to genetic counseling for evaluation. Genetic testing was recommended on 30 and completed on 23. Six mutation carriers and the patient with a somatic BRCA1 mutation were not referred to genetic counseling due to perceived low risk because they were older than 45 years old or did not have a first-degree family member with breast or ovarian cancer (adequate family size).

Information on BRCA status is now important not only to address the risk of breast and ovarian cancer, but also to select therapies. BRCA1 and BRCA2 play a critical role in DNA repair by homologous recombination. Poly (ADP-ribose) polymerase-1 (PARP1) inhibitors demonstrated synthetic lethality with BRCA1/BRCA2 dysfunction in homologous recombination deficient breast cancers and have shown efficacy as single agents in clinical trials in germline BRCA mutation carriers. The frequency of somatic BRCA1/2 mutations and expression loss are sufficiently common in ovarian cancer to warrant assessment of tumors in addition to germline DNA for patient selection for clinical trials of PARP1 inhibitors. On the other hand, somatic mutations were rare in TNBC with only one somatic mutation identified in 77 patients. However, our unselected patient cohort of TNBC shows a 19.5% incidence of BRCA deleterious mutations and almost half of the mutation carriers were not referred or tested mostly due to insufficient documented risk such as older age and lack of first degree relatives or insurance difficulties. Further, in recent work from our institution to estimate the costs and benefits of different BRCA testing criteria for women with breast cancer under age 50 using a Markov Monte Carlo simulation comparing six reference criteria for BRCA testing showed that testing women with triple-negative breast cancers under age 50 was the most cost-effective strategy and could reduce future breast and ovarian cancer cases by 26% and 45%, respectively, compared to the reference strategy.

Mutations of BRCA1/2 in TNBC were associated with better RFS after surgery and anthracycline and taxane-based chemotherapy, (p=0.031). After adjustment for other patient characteristics, patients with BRCA mutations had a significantly better RFS(HR: 0.19, 95% CI: 0.045-0.79, p=0.016) compared to no mutation carriers.

Although small, our unselected patient cohort of TNBC shows an important incidence of deleterious BRCA mutations suggesting that genetic testing should be discussed with patients with TNBC. Also, our results show that BRCA status is it is predictive of benefit from the systemic therapy regimens used in this patient cohort. It also suggests that testing either tumor or germline DNA of patients with TNBC is likely to identify a number of patients that could potentially benefit from PARP inhibitor therapy that would not be selected based on current BRCA mutation testing approaches based on family history.

### Example 2

BRCA1 and BRCA2 play a critical role in DNA repair by homologous recombination (HR). BRCA1 and BRCA2 germline mutations occur in 11-15.3% of women with ovarian cancer. Poly (ADP-ribose) polymerase-1 (PARP1) inhibitors are synthetic lethal with BRCA1 and BRCA2 dysfunction in HR-deficient cancers and are currently in clinical trials in BRCA1/2 germline mutation carriers with ovarian and breast cancer. The preliminary results of these clinical studies are encouraging. As PARP1 inhibitors may also be effective in cancers where BRCA1 or BRCA2 and thus HR function is compromised by somatic aberrations, the number of women with ovarian cancer who might benefit from PARP1 inhibitors may be greater than predicted by the frequency of germline BRCA1/2 mutations alone. However, the status of BRCA1 and BRCA2 has not been comprehensively studied in a large cohort of human ovarian cancers to assess whether loss of BRCA function can also occur due to somatic events. With this in mind, we evaluated BRCA1/2 in 235 unselected human ovarian cancers by sequencing BRCA1 and BRCA2, identifying intra-genic deletions using ultradense (20 base pair probe spacing) tiling arrays, determining gene copy number using 500K single nucleotide polymorphism (SNP) arrays, and quantifying expression of BRCA1/BRCA2 using quantitative-polymerase chain reaction (qPCR). Germline mutation status was determined where normal DNA could be obtained.

Methods

Patient Characteristics.

Unselected human ovarian cancer tissues (235) were obtained from the Gynecology Cancer Banks at M.D. Anderson Cancer Center (MDACC) and University of California San Francisco (UCSF) under Institutional Review Board (IRB)-approved protocols. The patient/cancer characteristics are shown in Table 6. As varying numbers of samples were used in the assays described in the following paragraphs, Table 3 lists the number of samples used in each assay and also gives the reason why only a subset of the 235 cancers were used in specific assays where applicable.

TABLE 6

| | |
|---|---|
| Total number of patients | 235 |
| Age at diagnosis | |
| Range | 23-92 |
| Median | 60 |
| Unknown | 20 (8.5%) |
| Follow-up time | |
| Range | 19-6241 days |
| Median | 1071 days |
| Unknown | 8 (3.5%) |
| Stage | |
| 1 | 11 (5%) |
| 2 | 14 (6%) |
| 3 | 156 (66%) |
| 4 | 33 (14%) |
| Unknown | 21 (9%) |
| Histology | |
| Serous | |
| Non-serous | 186 (79%) |
| Mixed | 13 (6%) |
| Unknown | 13 (6%) |
| Grade | 22 (9%) |
| 1 | 13 (5.5%) |
| 2 | 19 (8%) |
| 3 | 180 (76.5%) |
| Unknown | 23 (10%) |
| Residual disease after surgery | |
| 0 | 12 (5%) |
| ≦1 cm | 126 (53.5%) |
| >1 cm | 60 (25.5%) |
| Unknown | 37 (16%) |
| Surgery | |
| Yes | 230 (98%) |
| No | 5 (2%) |
| Unknown | |
| Chemotherapy | |
| No chemotherapy | 9 (3.8%) |
| Unknown | 33 (14%) |
| Platinum (cis or carboplatin)-based (no | 17 (7.2%) |
| taxane) | |
| Platinum plus Taxane (paclitaxel or | 176 (74.9%) |
| docetaxel)-based | |

TABLE 7

| | | If the assay was not applied |
|---|---|---|
| | Number of | to all 235 ovarian cancer cases, |
| Assay | samples | the reason for this was as follows: |
| Sequencing- | 235 | — |
| BRCA1/BRCA2 | | |
| Sequencing-TP53 | 113 | Samples were selected randomly |
| | | so as to determine if an |
| | | association existed between |
| | | TP53 and BRCA1/2 mutations |
| High-Density Tiling | 65 | Samples were selected |
| Array-BRCA1/BRCA2 | | randomly and the assay |
| | | was discontinued due to low yield |
| | | (only 1/65 intragenic deletions |
| | | (in BRCA1) was detected) |
| Quantitative-PCR of | 235 | — |
| BRCA1 and BRCA2 | | |
| expression | | |
| Affymetrix 500K SNP | 203 | Samples with sufficient DNA |
| arrays | | remaining after completion of |
| | | the assays listed above were used |

RNA/DNA Extraction from Frozen Cancers.

10 μm thick sections from frozen cancer blocks in Tissue-Tek OCT (Qiagen, Valencia, Calif.) were homogenized using a TissueRuptor (Qiagen) after adding QIAzol lysis reagent, followed by RNA isolation using a QIAgen miRNAeasy Mini Kit per manufacturers protocol. A QIAamp DNA Mini Kit (QIAgen) was used to isolate DNA per the manufacturer's protocol with overnight incubation at 56° C. and RNaseA treatment.

Quantitative-PCR.

Reverse transcription was performed using a High-Capacity cDNA Reverse Transcription Kit (Applied Biosystems, Inc.) per manufacturer instructions. For pre-amplification, a 0.2× probe mix was made by combining 1 uL of 91 20X gene expression assays from Applied Biosystems Inc. and 9 uL of low-EDTA TE. Pre-amplification was performed using 2.5 uL of 2× TaqMan° PreAmp Master Mix (Applied Biosystems, Inc), 1.25 uL of 0.2× probe mix, and 1.25 uL cDNA. Applied Biosystems TaqMan assays (BRCA1: Hs00173233_m1, Hs00173237_m1, Hs01556190_ml, Hs01556191_m1; BRCA2: Hs00609060_m1; housekeepers: Hs99999908_m1 (GUSB), Hs00188166_m1 (SDHA), Hs00237047_m1 (YWHAZ), Hs00824723_m1 (UBC), Hs00609297_m1 (HMBS)) were used for pre-amplification and qPCR on a Fluidigm (South San Francisco, Calif.) BioMark instrument. Cycle conditions were 9° C. for 10 minutes, 17 cycles of 9° C. for 15 seconds and 6° C. for 4 minutes. The PCR products were diluted 1:5 with low-EDTA TE. Samples were assessed on gene expression M48 dynamic arrays (Fluidigm) per manufacturer's protocol. The comparative Ct method was used to calculate relative gene expression using the Ct for the BRCA2 assay, the average Cts from the BRCA1 assays, and the average Cts from housekeeper genes. qPCR was performed in 235 cancers.

Mutation Screening.

PCR was performed on 2 ng DNA in a 3 uL reaction using the primers flanking the exons of BRCA1/BRCA2 that are used in the BRCAnalysis® (Myriad Genetics, Salt Lake City, Utah) clinical test with the following cycling conditions: 9° C. for 10 minutes, 35 cycles of 9° C. for 30 seconds, 6° C. for 30 seconds and 7° C. for 1 minute, finishing with 7° C. for 1 minute. Each PCR product was treated with 0.1 U Shrimp Alkaline Phosphatase (Sigma-Aldrich Inc.) The PCR product was diluted 1:9 and 0.8 uL was used for cycle sequencing with Big Dye Sequencing Chemistry and Taq FS (Applied Biosystems Inc.). Cycle conditions were 9° C. for 3 minutes, 32 cycles of 9° C. for 30 seconds, 5° C. for 30 seconds, 6° C. for 3 minutes, finishing with 7° C. for 10 minutes. Sequence products were run on a Megabace 4500 automated sequencer (GE) according to the manufacturer's protocol.

TP53 was amplified in 113 cancers using nested PCR. Primary PCR was performed using Taq-Platinum and 1 ul of 2 ng/ul DNA in a 3 ul reaction with primers without M13 tails. Cycle conditions were 96° C. for 5 minutes, 24 cycles of 95° C. for 20 seconds, 55° C. for 30 seconds, 72° C. for 2 minutes, followed by 72° C. for 10 minutes. This PCR product was diluted 9-fold and used for a secondary reaction with primers that have M13 tails. Cycle conditions were the same as the primary. Sequence products were run on a Megabace 4500 automated sequencer (GE) according to the manufacturer's protocol.

High-Density Tiling Array.

The array was designed using eArray (Agilent Technologies) and synthesized on a 8×15000 probe format. The array design included probes spaced at 20 by intervals across the complete genomic region of 2 genes (BRCA1/BRCA2) from 10 kb upstream of the 5′UTR to 5 kb downstream of the 3′UTR avoiding repeats. Additional probes (1000) were evenly distributed across the genome to form a backbone against which specific genomic gain/loss was estimated.

Sample preparation/array processing was performed using the Oligonucleotide Array-Based CGH for Genomic DNA Analysis kit and protocol (Agilent Technologies). These arrays were run on 65 ovarian cancers. Data was analyzed using DNA Analytics 4.0 (version 4.0.76) software (Agilent Technologies).

Affymetrix 500K SNP Arrays.

These arrays were run on 203 cancers. 250 ng genomic DNA was processed using Affymetrix GeneChip Mapping NspI or Styl Assay Kit as per the manufacturers protocol and hybridized to Affymetrix Mapping 500K NspI or Styl microarrays. After hybridization, array wash, stain and scan procedures were performed per the manufacturer's protocol. Copy number and LOH analysis were performed using a software package described elsewhere (manuscript in preparation). Only chips with high-quality data were used for the final analysis.

Statistical Analysis.

All analyses were carried out using R, version 2.9.0 (www.R-project.org). In all analyses, observations were removed if the response or a covariate was missing. Fisher's exact test was used to make comparisons involving pairs of categorical variables. Student's t-tests were used to compare differences in means of continuous variables. Cox's proportional hazards regression was used to perform univariate and multivariate analysis on the progression free survivial (PFS) and overall survival (OS) times from the date of debulking surgery. Comparisons of survival probabilities for categorical variables were visualized with Kaplan-Meier plots. The partial likelihood ratio test was used to compute p-values. Wald statistic-based confidence intervals (CI) were calculated for hazard ratio (HR) point estimates.

Results

BRCA1 and BRCA2 Mutations in Ovarian Cancers.

In DNA extracted from 235 human ovarian cancers (Table 6), 45 mutations in BRCA1 (32) and BRCA2 (13) were detected including two small homozygous intra-genic BRCA1 deletions that were detected using tiling arrays. This equates to a BRCA1/BRCA2 mutation frequency of 18.7, higher than the expected frequency of germline mutations in an unselected population of ovarian cancer patients. When confined to high-grade (grade3) serous cancers, the frequency of BRCA1/BRCA2 mutations was 23.4% (37/158).

Germline vs. Somatic BRCA1/BRCA2 Mutations in Ovarian Cancers.

Of the 44 patients with ovarian cancers harboring a BRCA1 or BRCA2 mutation, germline DNA was available from 28. In these 28 patients, 11(39.3%, CI=(22.1, 59.3)) ovarian tumor BRCA1 and BRCA2 mutations could be demonstrated to be somatic due to an inability to detect the aberration in germline DNA, while 17 (60.7%) mutations were found in both tumor and germline DNA. For BRCA1 and BRCA2, 9/21 (42.9%) and 2/7 (28.6%) mutations were somatic, respectively.

Homozygous BRCA1/BRCA2 Deletions in Ovarian Cancers.

One homozygous intra-genic deletion in BRCA1 and none in BRCA2 was detected by high-density tiling arrays in 65 ovarian cancers. Given this low frequency, tiling arrays were not run in all 235 cancers. Homozygous deletion of both copies of BRCA1 or BRCA2 was not detected by 500K SNP array, confirming the low frequency of deletions in tumors. A second homozygous intra-genic deletion of the same exon in BRCA1 was detected by haplotype analysis of the sequencing data, and confirmed using a high-density tiling array.

Loss of Heterozygosity (LOH) of BRCA1/BRCA2 in Ovarian Cancers.

LOH in BRCA1 was detected in 86/98 (87.8%) ovarian cancers. In contrast, LOH in BRCA2 was detected in significantly fewer (46/89 (51.7%); p<0.0001) ovarian cancers. The one retained gene copy was duplicated (a phenomenon known as copy neutral LOH) in 27/46 cases of LOH of BRCA2 (58.7%) and 38/86 cases of LOH at BRCA1 (44.2%). Interestingly, LOH of BRCA2 was only detected in one of the samples without LOH of BRCA1 (p=0.001).

We also hypothesized that loss of expression of BRCA1 or BRCA2 in ovarian cancer would, as with BRCA1 or BRCA2 mutations, impair the function of BRCA1 or BRCA2 and thus lead to significantly improved PFS times after surgery and platinum-based chemotherapy. Cancers without BRCA1/2 mutations were considered to have loss of BRCA1 and/or BRCA2 expression if the average of BRCA1 or BRCA2 dCT was higher than the 95th percentile of a normal distribution fit to the mutants' dCT. Loss of BRCA1 and/or BRCA2 expression was present in 24 (14%) BRCA1/2-wild type cancers, implicating other potential mechanisms (e.g. methylation) in loss of BRCA1 and BRCA2 gene expression.

Survival Associations.

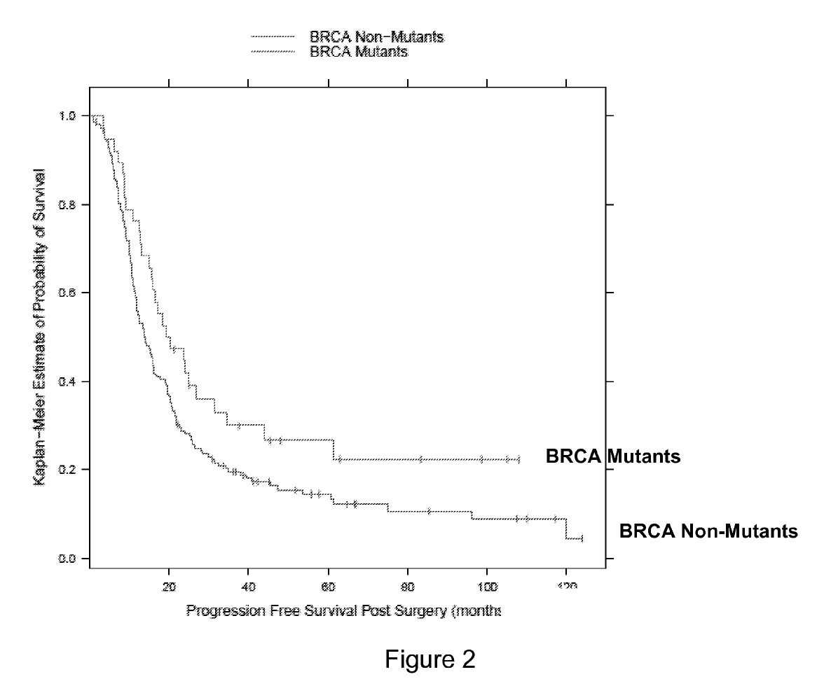

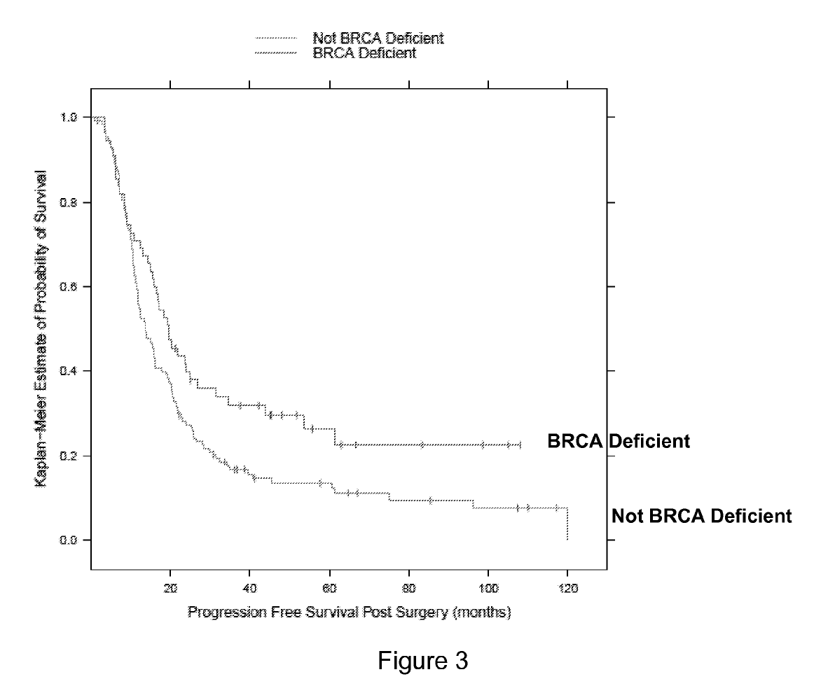

Previously, germline mutations in BRCA1 and BRCA2 have been reported to be associated with improved outcomes for ovarian cancer patients after surgery and platinum-based chemotherapy. 8-10 Likewise, herein, BRCA1 and BRCA2 mutations in ovarian cancer tissue together were associated with a significantly improved PFS as compared with BRCA1- and BRCA2-wild type cancers in univariate analysis (FIG. 2; p=0.041 (HR=0.67, CI=(0.44, 1.00)), n=195). This significant association was maintained in a multivariable Cox model that included clinical variables (Table 8). PFS for individuals with germline BRCA mutations was not significantly different from individuals with somatic BRCA mutations (p=0.362). An integrated dichotomous BRCA1/2-deficiency variable was thus defined as the presence of mutations in or loss of gene expression of BRCA1 or BRCA2. This BRCA1/2 deficiency variable was present in 65/220 (29.6%) (ovarian cancers. In univariate analyses, BRCA1/2 deficiency was significantly associated with PFS (FIG. 3; p=0.021 (HR=0.66, CI=(0.46, 0.95)), n=184). This significant association of BRCA1/2 deficiency with PFS was maintained in a multivariable Cox model (Table 5).

TABLE 8
A multivariable Cox model of progression-free survival
(PFS) in women with ovarian cancer (n = 170).

| | | Hazard Ratio |
|---|---|---|
| Variable | p-value | (95% CI) |
| Residual disease | 0.002 | 1.81 (1.25, 2.61) |
| Stage | 0.002 | 2.38 (1.27, 4.46) |
| Grade | 0.028 | 1.75 (1.03, 2.98) |
| BRCA1/2 mutation | 0.028 | 0.63 (0.41, 0.97) |
| status | | |

Residual disease was a binary variable (0 for 1 cm or less and 1 for greater than 1 cm). Stage was a binary variable (0 for stages 1 and 2 and 1 for stages 3 and 4). Grade was a binary variable (0 for grades 1 and 2 and 1 for grade 3).

TABLE 9
Multivariable Cox models of progression-free survival
(PFS) in women with ovarian cancer (n = 156).

| | | Hazard Ratio |
|---|---|---|
| Variable | p-value | (95% CI) |
| Residual disease | 0.002 | 1.91 (1.30, 2.80) |
| Stage | 0.004 | 2.30 (1.23, 4.31) |
| Grade | 0.017 | 1.85 (1.07, 3.20) |
| BRCA1/2 deficiency | 0.006 | 0.59 (0.40, 0.87) |

Residual disease was a binary variable (0 for 1 cm or less and 1 for greater than 1 cm). Stage was a binary variable (0 for stages 1 and 2 and 1 for stages 3 and 4). Grade was a binary variable (0 for grades 1 and 2 and 1 for grade 3).

Associations Between BRCA1 and BRCA2 and Other Mutations.

The mutation status of TP53 was available for 113 ovarian cancers. TP53 mutations were present in 81 (71.7%) cases and were significantly associated with BRCA1 and all BRCA1/2 mutations (Table 10). Given the strong association of mutations in BRCA1 and of both BRCA1/2 mutations with TP53 mutations, we hypothesized that if LOH represented loss of BRCA function, it should also be associated with TP53 mutations. Indeed, we found that TP53 mutations were significantly associated with LOH in BRCA1 (54/63 vs. 0/10; p<0.0001) and marginally associated with LOH in BRCA2 (28/34 vs. 18/30; p=0.057).

TABLE 10

| | TP53 | TP53 non- | |
|---|---|---|---|
| | mutant | mutant | p value |
| BRCA1 mutant | 20 | 1 | |
| BRCA1 non- | 61 | 31 | 0.007 |
| mutant | | | |
| BRCA2 mutant | 4 | 2 | |
| BRCA2 non- | 77 | 30 | 1 |
| mutant | | | |
| BRCA mutant | 23 | 3 | |
| BRCA non- | 58 | 29 | 0.045 |
| mutant | | | |

### Discussion

This is the first comprehensive study of BRCA1/2 status in ovarian cancer tissue. Although thought previously to be uncommon, we have demonstrated that somatic mutations in the BRCA1/2 genes account for at least one-third of BRCA1 and BRCA2 mutations in ovarian cancer specimens. In fact, BRCA1/2 mutations in total occur in approximately 19% of all ovarian cancers and in approximately 23% of high-grade serous ovarian cancers, compared to previous reports that BRCA1 and BRCA2 germline mutations occur in 11-15.3% of unselected women with ovarian cancer. Based on our germline sequencing, we estimate a germline mutation rate of approximately 13.5% in our dataset and a somatic mutation rate of 5.5%. Mutations of BRCA1 and BRCA2 in ovarian cancers are associated with improved PFS times after surgery and platinum/taxane-based cytotoxic chemotherapy, likely as a result of impaired HR in cancers with BRCA1/2 mutations. This is consistent with several previous reports for germline BRCA1- and BRCA2 mutations in women with ovarian cancer and likely represents, at least in part, increased effectiveness of platinum drugs in cancer cells with deficient HR. We also hypothesized that loss of expression of BRCA1 or BRCA2 in ovarian cancer would, as with BRCA1 or BRCA2 mutations, impair the function of BRCA1 or BRCA2 and thus lead to significantly improved PFS times after surgery and platinum-based chemotherapy. Indeed, BRCA1/2 deficiency (mutations plus expression loss) was significantly associated with PFS, suggesting that loss of BRCA1- and BRCA2 expression likely occurs for reasons other than mutations and homozygous deletions and may also impair HR in cancer cells. Of note, the numbers of low BRCA1/2 expressors in our study were not consistent with reported rates of methylation of these genes in the literature (approximately 20%). Further, homozygous BRCA1/2 deletions were rare in our study.

Mutations of BRCA1 are almost universally associated with TP53 mutations. This is consistent with genetically engineered mouse models in which BRCA1 deletion is lethal whereas embryos with combined BRCA1 and TP53 mutations survive significantly longer.

Currently, screening for germline BRCA1 and BRCA2 mutations is performed in women with ovarian cancer who are judged to be at high risk for carrying an inherited mutation based on clinical models (e.g., BRCAPRO). These models utilize factors including family history and age at diagnosis to establish risk. PARP1 inhibitor trials are currently underway in BRCA1/2 germline mutation carriers with ovarian cancer and the preliminary results of these studies are encouraging. However, since PARP1 inhibitors are selectively active in BRCA1 and BRCA2-deficient cancers, then assessment of BRCA1/BRCA2 mutation status in all ovarian cancers could identify a higher number of women who might benefit from these novel drugs.

In summary, loss of BRCA function due to frequent somatic aberrations in ovarian cancers likely deregulates HR and thereby increases sensitivity to platinum drugs and possibly also to novel PARP1 inhibitors. This is consistent with prior studies of loss of BRCA function due to germline mutations. The novelty of our findings is the observation that somatic BRCA/1 gene aberrations occur frequently and this observation may significantly increase the number of patients who will benefit from PARP1 inhibitors in ovarian cancer clinical trials. Somatic and germline mutations as well as BRCA1/2 expression loss are sufficiently common in ovarian cancer to warrant assessment in clinical trials for prediction of benefit from PARP1 inhibitors.

It is specifically contemplated that any embodiment of any method or composition of the invention may be used with respect to any other method or composition of the invention.

In the context of genes and gene products, the name of the gene is generally italicized herein following convention. In such cases, the italicized gene name is generally to be understood to refer to the gene (i.e., genomic), its mRNA (or cDNA) product, and/or its protein product. Generally, though not always, a non-italicized gene name refers to the gene's protein product.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

Unless otherwise defined, all technical and scientific terms used herein have the same meaning as commonly understood by one of ordinary skill in the art to which this invention pertains. In case of conflict, the present specification, including definitions, will control. In addition, the materials, methods, and examples are illustrative only and not intended to be limiting.

Other features and advantages of the invention will be apparent from the preceding detailed description and from the following claims

1. A method comprising determining whether a patient's tumor sample has BRCA deficiency and, if there is somatic BRCA deficiency, determining whether the patient has germline BRCA deficiency.

2. A method comprising identifying a patient having TNBC, determining whether a patient's tumor sample has BRCA deficiency and, if there is somatic BRCA deficiency, determining whether the patient has germline BRCA deficiency.

3-4. (canceled)

5. The method of claim 1, wherein said patient is identified as lacking any significant risk factors for germline BRCA deficiency.

6. The method of claim 5, wherein said patient is identified as lacking any significant personal or family history of cancer.

7. The method of claim 5, wherein said patient lacks any of the following: breast cancer at ≦40 years, bilateral breast cancer; breast cancer at ≦50 years and a close relative with breast cancer at ≦50 years; Ashkenazi Jewish ancestry with breast cancer at ≦50 years; breast or ovarian cancer at any age and two or more close relatives with breast cancer at any age; a first or second degree relative that meets at least one of the above criteria.

8. (canceled)

9. The method of claim 1, wherein said patient's cancer was diagnosed after 55 years of age.

10-12. (canceled)

13. A method of treating a patient comprising determining whether a patient has TNBC, determining whether the patient is BRCA defective, and, if the patient is BRCA defective, selecting a treatment course selected from the group consisting of: (a) treatment comprising a DNA-damaging agent, (b) treatment comprising a PARP inhibitor, (c) treatment comprising prophylactic mastectomy, (d) treatment comprising oophorectomy, and (e) treatment comprising prophylactic pharmaceutical treatment.

14-18. (canceled)

19. The method of claim 2, wherein said patient is identified as lacking any significant risk factors for germline BRCA deficiency.

20. The method of claim 19, wherein said patient is identified as lacking any significant personal or family history of cancer.

21. The method of claim 19, wherein said patient lacks any of the following: breast cancer at ≦40 years, bilateral breast cancer; breast cancer at ≦50 years and a close relative with breast cancer at ≦50 years; Ashkenazi Jewish ancestry with breast cancer at ≦50 years; breast or ovarian cancer at any age and two or more close relatives with breast cancer at any age; a first or second degree relative that meets at least one of the above criteria.

22. The method of claim 19, wherein said patient's cancer was diagnosed after 55 years of age.

23. A system for detecting BRCA deficiency comprising: (1) a sample analyzer for determining the sequence of BRCA1 or BRCA2 in a sample from a patient, wherein said patient has TNBC and wherein the sample analyzer contains the sample, genomic BRCA1 or BRCA2 DNA from said sample, or DNA synthesized from said genomic BRCA1 or BRCA2 DNA; (2) a first computer program for determining whether the sample has a BRCA deficiency by comparing the sequence determined by the sample analyzer to a reference BRCA1 or BRCA2 sequence; and (3) a display means for displaying whether the patient has BRCA deficiency.

24. The method of claim 23, wherein said patient is identified as lacking any significant risk factors for germline BRCA deficiency.

25. The method of claim 24, wherein said patient is identified as lacking any significant personal or family history of cancer.

26. The method of claim 24, wherein said patient lacks any of the following: breast cancer at ≦40 years, bilateral breast cancer; breast cancer at ≦50 years and a close relative with breast cancer at ≦50 years; Ashkenazi Jewish ancestry with breast cancer at ≦50 years; breast or ovarian cancer at any age and two or more close relatives with breast cancer at any age; a first or second degree relative that meets at least one of the above criteria.

27. The method of claim 24, wherein said patient's cancer was diagnosed after 55 years of age.
